(19) United States
(12) Patent Application Publication
(10) Pub. No.: US 2009/0068164 A1
(43) Pub. Date: Mar. 12, 2009

(54) SEQUENCE ENABLED REASSEMBLY (SEER) - A NOVEL METHOD FOR VISUALIZING SPECIFIC DNA SEQUENCES

(75) Inventors: David J. Segal, Davis, CA (US); Indraneel Ghosh, Tucson, AZ (US); Aik T. Ooi, Tucson, AZ (US); Jason Porter, Tucson, AZ (US); Cliff L. Stains, Tucson, AZ (US); Carlos F. Barbas, Solana Beach, CA (US)

Correspondence Address: OBLON, SPIVAK, MCCLELLAND MAIER & NEUSTADT, P.C., 1940 DUKE STREET, ALEXANDRIA, VA, 22314, US

(73) Assignees: THE ARIZ BD OF REGENTS ON BEHALD OF THE UNIV OF AZ, Phoenix, AZ (US); THE SCRIPPS RESEARCH INSTITUTE, La Jolla, CA (US)

(21) Appl. No.: 11/913,592

(22) PCT Filed: May 5, 2006
(86) PCT No.: PCT/US2006/017425

§ 371 Date: Oct. 23, 2008

Related U.S. Application Data
(60) Provisional application No. 60/678,453, filed on May 5, 2005.

(51) Int. Cl. A61K 38/46; C12Q 1/68; A61P 35/00; C07H 21/04

(52) U.S. Cl. 424/94.6; 435/6; 536/23.1

(57) ABSTRACT

The present invention provides a nucleotide sequence detection system in which a reporter enzyme is split into two halves each half of which is associated with at least one zinc finger domain. Upon DNA binding to the specific sequence defined by the zinc finger domains associated with the respective halves, the split-protein reassembles to reconstitute a functional enzyme. As such, the present invention provides methods of using the nucleotide sequence detection system for various diagnostic and identification purposes.

### CROSS-REFERENCE TO RELATED APPLICATIONS

The present application claims priority to U.S. Application No. 60/678,453, filed on May 5, 2005, which is hereby incorporated by reference in its entirety.

### STATEMENT REGARDING FEDERALLY SPONSORED RESEARCH

This work was supported by the National Institutes of Health Grant No. NIH/NIGMS; T32 GM008804. The United States government is entitled to certain rights in the present application.

### BACKGROUND OF THE INVENTION

1. Field of the Invention

The present invention provides a nucleotide sequence detection system in which a reporter enzyme is split into two halves each half of which is associated with at least one sequence-specific DNA-binding domain. Upon DNA binding to the specific sequence defined by the sequence-specific DNA-binding domains associated with the respective halves, the split-protein reassembles to reconstitute a signal generating protein. As such, the present invention provides methods of using the nucleotide sequence detection system for various diagnostic and identification purposes.

2. Discussion of the Background

In eukaryotes, a complex set of regulatory elements control the initiation of transcription of genes. Corresponding sequence-specific binding proteins recognize specific nucleic acid sequences. These proteins are called transcription factors. Transcription factors have at least two functionally different domains, one that binds to a specific nucleic acid sequence and another that activates or represses transcription. A major class of transcription factors called zinc fingers (ZFPs) are proteins that contain at least one zinc finger and can bind specifically to nucleic acids in a sequence specific mariner. The zinc finger proteins were first described in 1985 in the transcription factor TFIIIA from the oocytes of the African clawed toad, Xenopus laevis.

The zinc finger motif is one of the most abundant DNA binding motifs. The most common and most widely used binding motif is the Cys2His2 family of zinc fingers. Proteins may contain more than one finger in a single chain and consisting of 2 anti-parallel beta-strands followed by an alpha helix. These proteins use a single zinc ion coordinated by 2 invariant histidine residues and 2 invariant cystine residues. Each of the zinc finger domains is capable of recognizing a 3-base pair tract in the major groove utilizing an alpha-helix. Thus a 3-finger protein can recognize a tract of 9 base pairs with picomolar to nanomolar affinity. The identification of a recognition code for nearly all-possible 3 base-pair recognition sites has allowed for the design of unique zinc fingers for nearly any nucleic acid target of interest. Zinc fingers vary widely in structure, as well as in function, which ranges from DNA or RNA binding to protein-protein interactions and membrane association. Other sequence specific transcription factor families, such as helix-turn-helix or designed DNA binding proteins can also serve as sequence specific DNA binding agents. Similarly, specific proteins also exist, containing methyl binding domains (MBD), that can recognize specific DNA methylation at CpG dinucleotide sequences that serve as another distinct class of DNA binding motifs.

A useful system for identifying macromolecular interactions is protein complementation assays (PCA). In these systems fragments of a detectable marker or reporter molecules, such as a protein, are used. When the fragments are proximate, a detectable signal is generated (U.S. Pat. No. 6,780,599, U.S. Pat. App. No. 20040229240 and U.S. Pat. App. No. 20030108869). Such systems include beta-lactamase, green fluorescent protein (GFP) and its many variants, dihydrofolate reductase, luciferase and beta-galactosidase.

Over the past few decades, medical clinicians and research scientists have uncovered the importance of genomic modifications and their role in the development of genetic abnormalities and cancer. Further, researchers have discovered the powerful tool that is DNA sequencing and marker identification. In addition to cancer and genetic screening (e.g., pre-natal or genetic counseling), DNA sequencing and marker detection has found tremendous utility in virtually all walks of life ranging from basic research to courts of law. In view of the ever expanding role for DNA-based technology, there exists a critical need for the development of new and efficient techniques based on site-specific DNA recognition that possess a broad range of applications, no less of which would be for clinical diagnostics.

### SUMMARY OF THE INVENTION

With the foregoing need in mind, the present invention provide herein novel constructs of DNA binding protein modules appended to split-protein reporter systems to detect specific nucleic acid sites of interest including specific sites of DNA methylation.

It is an object of the present invention to provide a general methodology for the direct detection of DNA by the design of a split-protein system that reassembles to form an active complex only in the presence of a targeted DNA sequence. This approach called SEquence Enabled Reassembly (SEER) of proteins combines the ability to rationally dissect proteins to construct oligomerization dependent protein reassembly systems and the availability of DNA binding Cys₂-His₂ zinc-finger motifs or other site-specific DNA-binding domains for the recognition of specific DNA sequences. In the Examples, the present inventors demonstrate the feasibility of the SEER approach utilizing the split Green Fluorescent Protein (GFP) appended to zinc finger domains, such that GFP chromophore formation is only catalyzed in the presence of DNA sequences that incorporate binding sites for both zinc fingers.

In another example of the present invention another SEER system is described that provides with catalytic capability using the split reporter enzyme TEM1 β-lactamase. As shown in the Examples, signal amplification remained linear over the assay time, and target DNA could be distinguished from non-target DNA in less than 5 minutes. A single base pair substitution in the DNA binding sequence reduced the signal to background levels. Substitution of a different custom zinc finger DNA-binding domain produced a signal only on the new cognate target.

A further example of the SEER approach can be utilized to detect specific sites of DNA methylation. In this approach a methyl binding domain (MBD) protein is utilized to target a methylated CpG dinucleotide and attached to one half of split GFP, whereas the other half of the split-GFP is attached to a sequence specific DNA binding domain, such as a zinc finger. In this example GFP chromophore formation is selectively catalyzed when a nucleotide sequence contains both a methylated CpG site as well as the zinc finger DAN binding site. These results present SEER as a rapid and sensitive method for the detection of double-stranded DNA sequences as well as specific sites of DNA methylation. The novel ability to read genetic information from double-stranded DNA provides several advantages over current detection methods.

(1) A nucleotide sequence detection system comprising:
a first protein wherein said protein comprises at least one sequence-specific DNA binding domain that may comprise a zinc finger domain or alternate DNA binding protein such as a helix-turn-helix protein, a miniature DNA binding protein, a methyl-cytosine binding domain, and the N-terminal oligomerization domain of a split-protein enzyme, wherein said at least one DNA binding domain (e.g., zinc finger domain) is separated from said N-terminal oligomerization domain of said split-protein enzyme by a linker; and
a second protein wherein said protein comprises at least sequence-specific DNA binding domain that may comprise a zinc finger domain or alternate DNA binding protein such as a helix-turn-helix protein, a miniature DNA binding protein, a methyl-cytosine binding domain, and the C-terminal oligomerization domain of said split-protein enzyme, wherein said at least one DNA binding domain (e.g., zinc finger domain) is separated from said C-terminal oligomerization domain of said split-protein enzyme by a linker.

(2) The nucleotide sequence detection system of (1), wherein said sequence-specific DNA binding domain is selected from the group consisting of a helix-turn-helix protein, a miniature DNA binding protein, a methyl-cytosine binding domain, and a zinc finger domain.

(3) The nucleotide sequence detection system of (2), wherein at least one of said first protein and said second protein contains at least one methyl-cytosine binding domain as said sequence-specific DNA binding domain.

(4) The nucleotide sequence detection system of (1), wherein at least one of said first protein and said second protein contains at least one zinc finger domain as said sequence-specific DNA binding domain.

(5) The nucleotide sequence detection system of (1), wherein each of said first protein and said second protein contain at least one zinc finger domain as said sequence-specific DNA binding domain.

(6) The nucleotide sequence detection system of (5), wherein said at least one zinc finger domain of said first protein is contained within a zinc finger module which is derived from a zinc finger protein selected from the group consisting of Zif268, PBSII and PE1A.

(7) The nucleotide sequence detection system of (5), wherein said at least one zinc finger domain of said second protein is contained within a zinc finger module which is derived from a zinc finger protein selected from the group consisting of Zif268, PBSII and PE1A.

(8) The nucleotide sequence detection system of (5), wherein said at least one zinc finger domain of said first protein are located C-terminal to the N-terminal oligomerization domain of said split-protein enzyme.

(9) The nucleotide sequence detection system of (5), wherein said at least one zinc finger domain of said second protein are located N-terminal to the C-terminal oligomerization domain of said split-protein enzyme.

(10) The nucleotide sequence detection system of (1), wherein said split-protein enzyme reassembles to form a functional enzyme;

wherein said first protein binds the cognate nucleotide sequence for the sequence-specific DNA biding domain comprised therein,

wherein said second protein binds the cognate nucleotide sequence for the sequence-specific DNA biding domain comprised therein, and

wherein the cognate nucleotide sequence for said first protein is located 5′ to the cognate nucleotide sequence for said second protein.

(11) The nucleotide sequence detection system of (10), wherein said split-protein enzyme is selected from the group consisting of beta-galactosidase, beta-lactamase, dihydrofolate reductase, green fluorescent protein and its variants, and luciferase.

(12) The nucleotide sequence detection system of (10), wherein said split-protein enzyme is a beta-lactamase.

(13) The nucleotide sequence detection system of (1), wherein said split-protein enzyme is green fluorescent protein and its variants.

(14) The nucleotide sequence detection system of (1), wherein said linker in said first protein ranges from 0 to 30 amino acids.

(15) The nucleotide sequence detection system of (1), wherein said linker in said first protein is 15 amino acids.

(16) The nucleotide sequence detection system of (1), wherein said linker in said second protein ranges from 0 to 30 amino acids.

(17) The nucleotide sequence detection system of (1), wherein said linker in said second protein is 15 amino acids.

(18) The nucleotide sequence detection system of (1), wherein said first protein has the sequence comprising SEQ ID NO: 16 and said second protein has the sequence comprising SEQ ID NO: 14.

(19) The nucleotide sequence detection system of (1), wherein said first protein has the sequence comprising SEQ ID NO: 46 and said second protein has the sequence comprising SEQ ID NO: 44.

(20) The nucleotide sequence detection system of (1), wherein said first protein has the sequence comprising SEQ ID NO: 48 and said second protein has the sequence comprising SEQ ID NO: 44.

(21) The nucleotide sequence detection system of (1), wherein said first protein has the sequence comprising SEQ ID NO: 16 and said second protein has the sequence comprising SEQ ID NO: 52.

(22) An isolated polynucleotide encoding said first protein of the nucleotide sequence detection system of (1).

(23) The isolated polynucleotide of (22), wherein said polynucleotide is selected from the group consisting of SEQ ID NO: 15, SEQ ID NO: 45, and SEQ ID NO: 47.

(24) An isolated polynucleotide encoding said second protein of the nucleotide sequence detection system of (1).

(25) The isolated polynucleotide of (24), wherein said polynucleotide is selected from the group consisting of SEQ ID NO: 13, SEQ ID NO: 43, and SEQ ID NO: 51.

(26) A kit comprising the nucleotide sequence detection system of (1) and a hybridization buffer.

(27) The kit of (26), wherein said first protein and said second protein are in a lyophilized form.

(28) A method of detecting the presence of a specific nucleotide sequence in a sample comprising a polynucleotide, wherein said method comprises:

contacting said sample with the nucleotide sequence detection system of (1) for a time and under conditions suitable to facilitate hybridization, wherein said nucleotide sequence detection system is tuned to detect said specific nucleotide sequence by the arrangement and number of sequence-specific DNA binding domains contained within said first protein and said second protein;

monitoring the formation of activity associated with the split-protein enzyme when in a reassembled state; and

correlating an observed positive activity from said monitoring to the presence of said specific sequence in said polynucleotide.

(29) The method of (28), wherein said split-protein enzyme is green fluorescent protein and said monitoring comprises monitoring the fluorescence emission at 509 nm upon excitation at 395 nm. Other GFP variants can be similarly utilized which have varying excitation and emission spectra.

(30) The method of (28), wherein said split-protein enzyme is beta-lactamase and said monitoring comprises monitoring hydrolysis of a substrate selected from the group consisting of nitrocefin, CCF2, CCF4, CC2, C-mel, penicillin, ampicillin, and carbonicillin.

(31) The method of (28), wherein said method is a method of detecting a genetic abnormality in a subject in need thereof.

(32) The method of (28), wherein said method is a method of detecting single nucleotide polymorphism in a subject in need thereof.

(33) The method of (28), wherein said method is a method of detecting shortening of telomeres in a subject in need thereof.

(34) The method of (33), wherein said subject in need thereof is a subject having or suspected of having cancer.

(35) The method of (34), wherein said subject in need thereof is a subject having or suspected of having an age related disease.

(36) The method of (28), wherein said method is a method of determining the age of cells or cloned animals and said specific nucleotide sequence is the repeat sequence in telomeres.

(37) The method of (28), wherein said method is a method of diagnosing cancer in a subject in need thereof and said specific nucleotide sequence is a unique marker for a specific type of cancer.

(38) The method of (28), wherein said method is a method of identifying an infectious agent and said sample is selected from the group consisting of a tissue sample, a blood sample, a sera sample, a nasal swab, a vaginal swab, and a rectal swab.

(39) The method of (28), wherein said method is a method of identifying an infectious agent and said sample is selected from the group consisting of food, beverage, and water.

(40) The method of (28), wherein said method is a sample-to-source matching method wherein the specific nucleotide sequence represents a unique nucleotide sequence obtained from a biological sample of interest and said sample is obtained from a subject suspected to contain said unique nucleotide sequence.

(41) The method of (40), wherein said biological sample of interest is selected from the group consisting of blood, hair, skin, sperm, and semen.

(42) The method of (40), wherein said sample is selected from the group consisting of blood, hair, skin, sperm, and semen.

(43) A method of treating eradicating a viral infection in a subject in need thereof, comprising:

tailoring the sequence specificity of said sequence-specific DNA binding domains of said nucleotide sequence detection system of (1) to the virus infecting said subject to a unique nucleic acid sequence thereto, wherein said split-protein enzyme facilitates hydrolysis of a substrate that becomes toxic to said virus upon hydrolysis;

administering an effective amount of said nucleotide sequence detection system of (1) to said subject; and

administering an effective amount of said substrate to said subject.

(44) The method of (43), wherein said split-protein enzyme is beta-lactamase.

(45) The method of (44), wherein said substrate is C-mel.

(46) A method of treating cancer in a subject in need thereof, comprising:

tailoring the sequence specificity of said sequence-specific DNA binding domains of said nucleotide sequence detection system of (1) to a mutant oncogene in said subject to a unique nucleic acid sequence thereto, wherein said split-protein enzyme facilitates hydrolysis of a substrate that becomes toxic to said virus upon hydrolysis;

administering an effective amount of said nucleotide sequence detection system of (1) to said subject; and

administering an effective amount of said substrate to said subject.

(47) The method of (46), wherein said split-protein enzyme is beta-lactamase.

(48) The method of (47), wherein said substrate is C-mel.

(49) A method of detecting the presence of specific sites of DNA methylation within a specific sequence of a polynucleotide of a subject in need thereof comprising:

tailoring the sequence specificity of said sequence-specific DNA binding domains of said nucleotide sequence detection system of (1) to a specific DNA sequence in said subject to a unique nucleic acid sequence thereto, wherein said sequence-specific DNA binding domain of at least one of said first protein and said second protein is a methyl binding domain;

delivering an effective amount of said nucleotide sequence detection system of (1) to a sample obtained from said subject;

monitoring the formation of activity associated with the split-protein enzyme when in a reassembled state; and

correlating an observed positive activity from said monitoring to the presence of DNA methylation within said specific sequence in said polynucleotide.

(50) The method of (49), wherein said methyl binding domain is a methyl-cytosine binding domain.

(51) The method of (50), wherein said first protein has the sequence comprising SEQ ID NO: 16 and said second protein has the sequence comprising SEQ ID NO: 52.

(52) The method of (49), wherein the presence of said DNA methylation is correlated with a propensity for or a diagnosis of cancer.

(53) A method for simultaneous detection the presence of multiple specific nucleotide sequences in a sample comprising a polynucleotide, wherein said method comprises:

contacting said sample with two or more different nucleotide sequence detection systems of (1) for a time and under conditions suitable to facilitate hybridization, wherein said nucleotide sequence detection systems are tuned to detect independent specific nucleotide sequences by the arrangement and number of sequence-specific DNA binding domains contained within said first protein and said second protein and wherein said split-protein enzyme for each nucleotide sequence detection system is distinct from any other,

monitoring the formation of activity associated with the split-protein enzymes when in a reassembled state; and

correlating an observed positive activity from said monitoring to the presence of said specific sequences in said polynucleotide.

(54) The method of (53), wherein said wherein at least one of said split-protein enzymes is selected from the group consisting of beta-galactosidase, beta-lactamase, dihydrofolate reductase, green fluorescent protein, and luciferase, and variants or homologs thereof.

(55) The method of (53), wherein at least one of said split-protein enzymes is a beta-lactamase, variants or homologs thereof.

(56) The method of (53), wherein at least one of said split-protein enzymes is green fluorescent protein, variants or homologs thereof.

(57) The method of (56), wherein at least one of said split-protein enzymes is selected from the group consisting of green fluorescent protein, cyan fluorescent protein, yellow fluorescent protein, red fluorescent protein, and reef coral fluorescent protein.

(58) The method of (53), wherein said contacting is with three to five of said nucleotide sequence detection systems.

The above objects highlight certain aspects of the invention. Additional objects, aspects and embodiments of the invention are found in the following detailed description of the invention.

### BRIEF DESCRIPTION OF THE FIGURES

A more complete appreciation of the invention and many of the attendant advantages thereof will be readily obtained as the same becomes better understood by reference to the following Figures in conjunction with the detailed description below.

FIG. 1 shows an overview of the SEER Strategy. NGFP-ZnFingerA comprises residues 1-157 of GFP fused by a 15-residue linker to the DNA binding zinc finger Zif268. CGFP-ZnFingerB comprises residues 158-238 of GFP fused by a 15-residue linker to the zinc finger PBSII.

FIG. 2. a) Fluorescence emission spectra of NGFP-ZnFingerA (15 μM)+CGFP-ZnFingerB (15 μM) in the presence and the absence of 4 μM target DNA (Zif268-10-PBSII) excited at 468 nm. Inset shows SDS-gel with mw standards (lane 1); equimolar mixture of NGFP-ZnFingerA and CGFP-ZnFingerB used in the SEER experiments (lane 2); NGFP-ZnFingerA (lane 3); and CGFP-ZnFingerB (lane 4). b) Fluorescence emission at 505 nm of NGFP-ZnFingerA (5 μM)+CGFP-ZnFingerB (5 μM) in the presence of indicated double stranded DNA controls (5 μM each). c) Relative fluorescence emission at 505 nm of NGFP-ZnFingerA (5 μM)+CGFP-ZnFingerB (5 μM) as a function of increasing concentrations of target DNA (Zif68-10-PBSII).

FIG. 3 shows a schematic of the pETDuet-SEER plasmid showing the position of the CGFP-PBSII and NGFP-Zif268 genes, restriction enzymes used, and T7 promoter sites.

FIG. 4 shows the fluorescence of SEER samples containing DNA with different spacing between binding sites.

FIG. 5 shows the configuration and orientation of LacA-Zif268 and PBSII-LacB constructs of Example 5.

FIG. 6 outlines the SEER-LAC strategy. LacA-Zif268 comprises residues 26-196 of β-lactamase fused by a 15-aa linker to the DNA binding ZF Zif268. PBSII-LacB comprises the ZF PBSII fused by a 15-aa linker to residues 198-290 of β-lactamase. In the presence of target DNA containing binding sites for Zif268 and PBSII with an appropriate spacer (0-bp spacer is shown), SEER fragments reassemble to form an active reporter enzyme.

FIG. 7 shows the DNA concentration-dependant SEER signal. A) Digital image of triplicate nitrocefin assays after 30 minutes incubation. DNA target oligonucleotides (with target site spacings of 0, 6 and 10 bp) and their concentrations are indicated above the image; SEER fragments (0.5 μM each) are indicated to the left. B) Graphical representation of the reaction kinetics for the assay shown in A. Absorbance at 486 nm was measured at 3 minutes and every 2 minutes after. Vmax (in milli-units/min) of the increase in absorbance was plotted. The negative control of non-cognate fragments (LacA-Zif268 & PE1A-LacB) was not shown, but had essentially the same signal as the “no DNA” negative control. C) Absorbance vs. time plot for LacA-Zif268 & PBSII-LacB (diamonds) and the non-cognate LacA-Zif268 & PE1A-LacB (triangles) with 1 μM Zif-0-PBSII DNA. A linear fit of the kinetic data (solid lines) confirmed a hydrolysis rate of 25.0 mU/min (R²=0.9951) and 3.4 mU/min (R²=0.9936), respectively. D) Reaction rate vs. DNA concentration plot for LacA-Zif268 & PBSII-LacB with Zif-0-PBSII DNA at 1 μM (lowest line of diamonds), 200 nM (top line of diamonds), and 200 pM (lowest line of diamonds).

FIG. 8 shows the sensitivity of SEER to mutations in the target DNA. A) Digital image of triplicate nitrocefin assays after 30 minutes incubation. A series of modified Zif-0-PBSII target oligonucleotides were used at 1 μM, containing 1, 2, 3 or 5 G to T substitutions (boxed) in either the Zif268 (left most 9 nucleotides on the left side and right side of the image) or PBSII target sites (right most 9 nucleotides on the left side and right side of the image), as indicated. SEER fragments LacA-Zif268 & PBSII-LacB were used at 0.5 μM each. B) Graphical representation of the reaction kinetics for the assay shown in A. Absorbance at 486 nm was measured at 3 minutes and every 2 minutes after.

FIG. 9 shows the SEER activity using various combinations of ZF binding domains and DNA targets. The Vmax of the reaction kinetics of triplicate nitrocefin assays is shown. Target oligonucleotides at 1 μM are indicated above the graph; SEER fragments at 0.5 μM each are indicated below.

FIG. 10 shows SEER binding in the presence of genomic DNA. LacA-Zif268 & PBSII-LacB at 0.5 μM each were incubated with 1 μM Zif-0-PBSII (dark bars) or 1 μM Zif-0-PE1A (light bars) for 20 minutes in the presence or absence (as indicated) of 3.2 μg of sheared, double-stranded Herring Sperm DNA. This concentration is equal in moles of base pairs (5.2 nmoles bp) to 1 μM of the target oligonucleotide.

FIG. 11 shows a schematic of the pETDuet CGFP-MBD2 plasmid of Example 7 showing the position of the CGFP-MBD2 gene and restriction enzymes used.

FIG. 12 shows an SDS Page for Example 8. MW standards (lane 1); NGFP-Zif268 (lane 2); CGFP-MBD2 (lane 3); and equimolar amounts of each protein (lane 4)

FIG. 13 shows the effect of target site spacing on SEER-GFP fluorescence as shown in Example 10.

### DETAILED DESCRIPTION OF THE INVENTION

Unless specifically defined, all technical and scientific terms used herein have the same meaning as commonly understood by a skilled artisan in enzymology, biochemistry, cellular biology, molecular biology, and the medical sciences.

All methods and materials similar or equivalent to those described herein can be used in the practice or testing of the present invention, with suitable methods and materials being described herein. All publications, patent applications, patents, and other references mentioned herein are incorporated by reference in their entirety. In case of conflict, the present specification, including definitions, will control. Further, the materials, methods, and examples are illustrative only and are not intended to be limiting, unless otherwise specified.

Virtually all scientific methods for reading the sequence information of DNA rely on the hybridization properties of complementary nucleic acid molecules. Such methods, including PCR, Sanger sequencing, DNA microarray, Southern and Northern blotting, and in situ hybridization, all consequently require denaturation of the native DNA double helix into single strands and subsequent renaturation with specific primers or probes under carefully controlled conditions. In contrast, nature frequently relies on sequence-specific DNA-binding proteins to read the sequence information of DNA, such as occurs during the processes of transcription initiation, intron homing, and defense against invasive DNA by restriction endonucleases. In the human genome, DNA-binding transcription factors comprise one of the largest classes of known genes, with approximately 2,000 members (19). The most common type of DNA-binding domain is the Cys2-His2 class of zinc fingers.

The present invention sets forth the development of a new technology for the detection of specific double-stranded (ds) DNA sequences. This system, designated SEER (SEquence-Enabled Reassembly), consists of split-protein systems that are able to reassemble an active complex only in the presence of a cognate DNA sequence. This approach merges two rational protein design approaches, the technology of protein complementation assays (PCA) and site-specific DNA-binding protein technology, for example custom zinc finger (ZF) protein technology.

PCA is a methodology initially described for detecting protein-protein interactions (14a,b). A functional protein, typically a reporter molecule, is dissected into two non-functional fragments. Functionality is restored when the fragments are reassembled by attached protein-protein interaction domains, such as leucine zippers. Several such systems have been recently reported, including reassemblies of β-galactosidase (14b), dihydrofolate reductase (DHFR) (14d), green fluorescent protein (GFP) and its variants (14e), TEM-1 β-lactamase (14c), and firefly luciferase (25, 26).

Sequence-specific DNA-binding proteins have been extensively studied over the past few decades. The present invention takes advantage of the wealth of information about the sequence specificity and the DNA-binding proteins responsible for that specificity. As such, the SEER constructs of the present invention provides two distinct protein constructs in which each construct contains at least one DNA binding protein/domain attached to one half of a protein from a PCA system.

Sequence-specific DNA binding proteins that may be used in the SEER system include, but are not limited to:

a) helix-turn-helix proteins; structural examples of this family of DNA binding proteins include those described by:
Huang G. S. et al. (1989) J. Mol. Biol. 205, 189-200. (ref. 33)
Mondragon, A. et al. (1989) J. Mol. Biol. 205, 179-188. (ref. 4′)
Neri D. et al. (1992) J. Mol. Biol. 223, 743-767. (ref. 40)
Pabo, C. O. et al. (1982) Nature 298, 443-447. (ref. 8)
Padmanabhan, S. et al. (1997) Biochemistry 36, 6424-6436. (ref. 22)
Sevilla-Sierra, P. et al. (1994) J. Mol. Biol. 235, 1003-1020 (ref. 42);

b) designed miniature DNA binding proteins, including those described in:
Yang L. & et al. (2005) Biochemistry, 44, 7469-7478. (ref. 20)
Montclare J. K. et al. (2003) J. Am. Chem. Soc., 125, 3416 (ref. 21);

c) zing finger proteins (below); and

d) methyl-cytosine binding domains, for example methyl-CpG binding domain family of proteins that includes MBD1, MBD2, MBD3, MBD4, and MeCP2 (47a,b).

Custom DNA-binding proteins can be constructed from modified Cys2-His2 ZF DNA-binding domains. Each ZF domain contains 30 amino acids that form a ββα fold, stabilized by hydrophobic interactions and the chelation of a zinc ion between two histidines and two cysteines. Each domain typically recognizes 3-4 nucleotides of DNA. The domains can be found in covalent tandem arrays, facilitating recognition of extended DNA sequences. A protein containing six zinc fingers should have the capacity to recognize 18-base pairs of DNA, sufficiently large to specify a unique site in the human genome (27).

A variety of combinatorial and rational design approaches have been used to modify the binding specificity of naturally occurring ZFs (28-31). In particular, Barbas and co-workers have produced a lexicon of interchangeable domains with the ability to recognize unique 3-4-base pair DNA sequences (15). Using these pre-defined recognition modules, DNA-binding proteins can be rapidly assembled to bind virtually any DNA-sequence or gene in the human genome (1). The three-finger proteins typically have affinities in the 1-50 nM range and are highly specific for their target site (27). These custom-made ZF proteins can be linked to functional domains to generate novel chimeric proteins that produce the desired activity at specific DNA sequences. This approach has been employed in designing targeted transcription factors (15, 34), targeted endonucleases (35), and targeted integrases (36). However, heretofore, no none methods or systems have been provided in which ZF proteins have been linked to a non-functional split-protein system that is able to reassemble an active complex only in the presence of a cognate DNA sequence.

Owing to the tunability of the DNA recognition site in the present invention, the SEER system of the present invention is a valuable tool to detect or confirm the presence of a particular a nucleic acid sequence, such as a genetic abnormality or a single nucleotide polymorphism (SNP). This system can be used to detect genomic rearrangements in DNA and for identification of highly repetitive sequences.

One example of an application for the present invention is identification of repeat sequences in telomeres. In humans, telomere sequences shorten over time producing ‘sticky’ end leading to chromosome rearrangements, which can be a marker for cancer or age related diseases. Since telomeres shorten with increasing age, detection of shortening telomeres can be useful, for example, to determine the age of cells or cloned animals.

Additionally, with respect to cancer and/or disease diagnostics, the SEER system of the present invention can be tailored to determine the absence or presence of a specific conserved sequence(s) that serves as a unique marker for the disease or type of cancer. The scope and identity of the genetic marker to be assayed is particularly limiting. As described herein, the nature and identity of the DNA binding protein and the sequence identified thereby may be selected by the skilled artisan depending upon the desired sequence to be detected.

Further, the present invention provides a method of detecting the presence of specific sites of DNA methylation within a specific sequence of a polynucleotide of a subject in need thereof by (a) tailoring the sequence specificity of said sequence-specific DNA binding domains of said nucleotide sequence detection system of claim 1 to a specific DNA sequence in said subject to a unique nucleic acid sequence thereto, wherein said sequence-specific DNA binding domain of at least one of said first protein and said second protein is a methyl binding domain, (b) delivering an effective amount of said nucleotide sequence detection system of claim 1 to a sample obtained from said subject, (c) monitoring the formation of activity associated with the split-protein enzyme when in a reassembled state, and (d) correlating an observed positive activity from said monitoring to the presence of DNA methylation within said specific sequence in said polynucleotide. Further, the presence of said DNA methylation can be correlated with a propensity for or a diagnosis of cancer. Of course, within this method it is contemplated that additional steps may be added including a sample recover step and any intermediate sample processing steps. The method of monitoring will vary depending upon the split-enzyme protein selected. In this method, the methyl binding domain is preferably a methyl-cytosine binding domain. In an embodiment of this method, the first protein has the sequence comprising SEQ ID NO: 16 and the second protein has the sequence comprising SEQ ID NO: 52.

In the foregoing applications, the sample to be assayed may be any cell containing sample. Non-limiting examples include tissue samples, including tissue biopsies, blood samples (including whole blood, red blood cells, or white blood cells), sera, nasal swabs, vaginal swabs, rectal swabs, etc.

SEER may also be used to make identification of other infectious agents such as virus (Ebola, Marburg, etc.), or identifying particular strains or serotypes of infectious agents such as HIV, Influenza or E. coli. In addition to the foregoing samples, the infectious agent may also be searched for in foods, beverages, water samples, etc.

SEER also finds application in the following areas of endeavor: a) detection of methylated DNA, reporting either the extent of methlyation or if a particular site is methylated in a cell, b) detection of DNA modified by environmental toxins, c) detection of DNA accessibility (e.g., reporting if a site on a chromosome is available to bind proteins or is protected by nucleosomes) or unusual DNA structures (e.g., G-quadruplex, triplex, cruciform), d) selection methodology as described below, e) therapeutic as described below.

In an embodiment of the present invention is a method of treating eradicating a viral infection or treating cancer in a subject in need thereof by tailoring the sequence specificity of the sequence-specific DNA binding domains of the SEER system to the virus infecting the subject or a mutant oncogene in the subject to a unique nucleic acid sequence thereto, wherein said split-protein enzyme facilitates hydrolysis of a substrate that becomes toxic to said virus upon hydrolysis, followed by administering to the subject an effective amount of the SEER system proteins and the substrate to be hydrolyzed. As stated below, an example of the split-protein enzyme for the SEER system that can effectuate this method is a beta-lactamase, where the substrate is C-mel. The term “effective amount” is meant to be an amount that brings about the desired therapeutic effect and will vary depending upon the age, weight, and condition of the subject, as well as the type of disorder to be treated or eradicated. In addition, the effective amount will vary on the basis of the cell type or target to be treated.

Additional applications in which the SEER system may be extended is in DNA profiling studies. The human genome comprises 3.2 billion base pairs and approximately 30,000 genes. As such, statistically, a unique site in the human genome can be defined by 16 consecutive nucleotides. For example, as stated above, a protein containing six zinc fingers should have the capacity to recognize 18-base pairs of DNA, which is sufficiently large to specify a unique site in the human genome. As such, by selecting a large enough DNA footprint the SEER system of the present application may be specifically tailored to detect the absence or presence of unique stretches of genomic DNA. This ability offered by the present invention provides for unique opportunities of sample-to-source matching based on DNA sequence, for example by comparative analysis of a stretch of DNA obtained from a blood, hair, skin, sperm, or semen sample (or other bodily fluids) recovered from a crime scene (or other more innocuous locale) with that of a DNA sample obtained from a suspect. An additional advantage provided by the SEER system in this application is that SEER could be implemented on-site. Typically, the amount of viable biological material (e.g., hair) recovered from the scene of a crime contains only a small quantity of DNA. Therefore, if the sample had to be collected and brought back to the lab for traditional PCR protocols, precious time and resources may be lost.

The use of SEER in intact cells is more advantageous than existing methods such as Fluorescent In Situ Hybridization (FISH). SEER will detect genetic difference and abnormalities between cells much like FISH. However, the SEER system allows for the detection of DNA accessibility, the presence of unusual DNA structures such as G-quadruplex and DNA modifications such as methylation, which are presently undetectable by FISH.

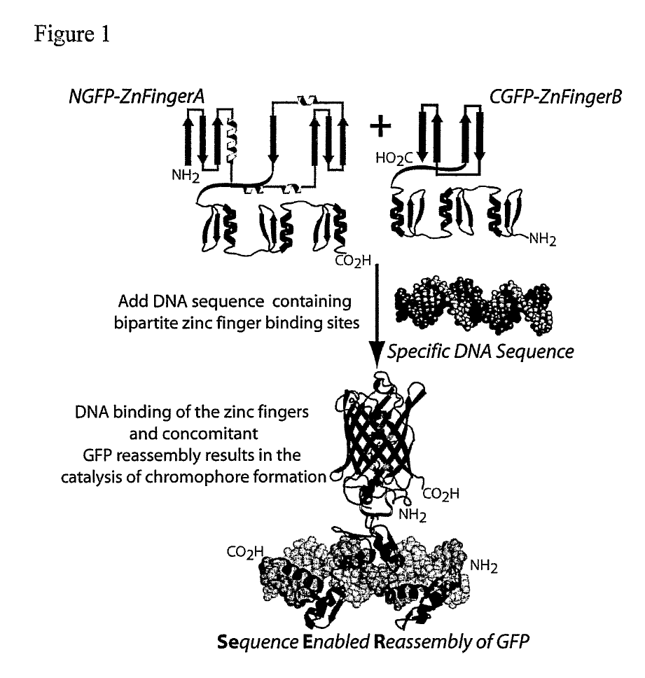

Oligomerization-assisted protein reassembly is possible when a protein can be fragmented into two halves that do not reassemble until appended to suitable protein oligomerization domains. This approach has been successfully utilized for the detection of oligomerizing proteins utilizing fragmented ubiquitin (14a), beta-galactosidase (14b), beta-lactamase (14c), dihydrofolate reductase (14d), green fluorescent protein (GFP) (14e,f), luciferase (14g), and PH domains (14h) among others. However split protein reassembly has not been utilized for the direct detection of specific DNA sequences by ternary complexation. In order for ternary complexation in the presence of DNA, we have chosen to employ the ubiquitous Cys₂-His₂ family of zinc fingers that are the most widely used DNA binding motif in the human genome. Each of the zinc finger domains is capable of recognizing a 3-base pair tract in the major groove utilizing an α-helix (16). Thus a 3-finger protein can recognize a tract of 9 base pairs with picomolar to nanomolar affinity (15). Moreover, recent experiments have resulted in the identification of a recognition code for nearly all-possible 3 base-pair DNA recognition sites, allowing for the design of unique zinc fingers for any DNA target of interest (15). The present inventors envision that appending sequence specific zinc fingers to appropriately fragmented proteins should in principle allow for protein reassembly only in the presence of the correct DNA sequence (FIG. 1).

Thus, the present invention provides a novel system to identify a desired nucleic acid sequence or, in the alternative, to determine the absence of a specific nucleic acid sequence that should exist, but due to mutation or modification is lost. This system utilizes pairs of specific hybrid proteins containing sequence-specific DNA binding domains or modules that bind to a polynucleic acid in a sequence specific manner. These hybrid proteins also include a PCA system fragment that when proximally located by the sequence-specific DNA binding domains or modules binding to nucleic acid generates the functional PCA reporter (FIG. 1).

In the case where the sequence-specific DNA binding domains or modules is one or more zinc finger protein, this system further utilizes known methods used to design custom site-specific nucleic acid-binding factors such as zinc finger proteins (1-4).

The zinc finger binding modules maybe derived from any known zinc finger protein including but not limited to Zif268 (residues 189-286 of SEQ ID NO: 44), PBSII (residues 5-88 of SEQ ID NO: 46) and PE1A (residues 5-88 of SEQ ID NO: 48). As stated above, a variety of combinatorial and rational design approaches have been used to modify the binding specificity of naturally occurring zinc fingers (28-31). In particular, Barbas and co-workers have produced a lexicon of interchangeable domains with the ability to recognize unique 3-4-base pair DNA sequences (15). As such, the zinc finger binding modules may be modified according to methods known in the art to bind a desired nucleic acid sequence (1-3). Additionally, zinc finger binding proteins may be assembled in multiples so as to define a recognition sequence of a length directly related to the number of zinc finger binding modules contained within the protein.

In an embodiment of the present invention one or both of the halves of the SEER system contain at least one helix-turn-helix protein, at least one designed miniature DNA binding protein, at least one methyl-cytosine (e.g., methyl-CpG) binding domain, and/or at least one zinc finger domain.

Therefore, in an embodiment of the present invention one or both of the halves of the SEER system contain at least one zinc finger domain. Preferably both halves of the SEER system contain at least one zinc finger domain, where the number of zinc finger domains can be asymmetrically distributed. The phrase “at least one zinc finger domain” embraces multiples defined only on the basis of the desired sequence to be detected. As such, the present invention embraces zinc finger domains in each half that are independently selected from 1, 2, 3, 4, 5, 6, 7, 8, 9, 10, etc.

Protein constructs were designed such that the protein fragment was fused to a an amino acid linker. Generally, this linker is about 15-residues. However, the linker length may be modified to increase flexibility and shortened to improve efficiency and selectivity (9). For example, the linker may be eliminated or shortened to at least 5 residues, preferably the linker is at least 10 residues. With respect to linkers of increased length, the following may be mentioned at least 20 residues, at least 25 residues, at least 30 residues. It should be apparent from the foregoing that the present inventors are in possession of and describe herein all integers falling within the ranges defined above although not specifically recited by number.

Previous studies using zinc finger based custom endonucleases have been used successfully in cells. The endonuclease systems relies on two zinc finger proteins to bring together catalytic domains of the restriction enzyme FokI at the appropriate target site to cleave DNA. These-protein pairs are successful in performing this function in cells from frogs, fly embryos, plants and humans (9-12).

The system of the present invention called sequence enabled enzyme reassembly (SEER) builds and expands upon the ability to rationally dissect enzymes to construct oligomerization-dependent protein reassembly systems and the ready availability of nucleic acid binding Cys2-His2 zinc-finger motifs for the recognition of desired nucleic acid sequences. Oligomerization-assisted protein reassembly is possible when a protein can be fragmented into two halves that do not reassemble until appended to suitable protein oligomerization domains. Previous systems have studied enzyme based detection system for protein-protein interactions.

The present inventors' design entailed choosing appropriate sequence-specific DNA binding domains (including, zinc fingers) and a suitable disassembled protein that could generate a readily detectable optical signal upon successful reassembly. For their disassembled protein set forth in Examples 1-4 below, they chose fragments of a GFP variant that have been previously demonstrated to be capable of functional reassembly only when appended to oligomerizing protein or peptide partners (14e, 17). It should also be noted that for the DNA binding domains in Examples 1-4, two well-characterized 3-domain containing zinc fingers Zif268 and PBSII, with low nanomolar affinity for unique 9-base pair sequences were chosen (15, 16). Further, in Examples 5-6 below, the inventors chose fragments of a beta-lactamase variant and demonstrate that this protein is capable of functional reassembly when appended to oligomerizing protein or peptide partners as reported by a colorimetric assay.

The SEER system is the first example of nucleic acid dependent reassembly of protein fragments, which can be applied to split-protein enzymes such as beta-lactamase, dihydrofolate reductase, green fluorescent protein, beta-galactosidase, and luciferase. Evidence for the enablement of this system is provided by the Examples of the present application wherein the SEER system has been applied using beta-lactamase and green fluorescent protein as split-protein enzymes. Thus, the SEER system defined in the present invention uses protein complementation assay systems that include, but are not limited to, beta-lactamase, dihydrofolate reductase, green fluorescent proteins, beta-galactosidase, and luciferase (5-7, 14e).

With respect to the green fluorescent protein, in addition to the exemplified green fluorescent protein, the present invention embraces all variants of the green fluorescent protein including. Further, the present invention also embraces structurally and functionally similar fluorescent proteins to the green fluorescent protein (i.e., variants and/or homologs of GFP), including reef coral fluorescent proteins, GFP variants such as Green, Cyan, Yellow, Red fluorescent proteins.

With respect to the GFP variants and the breadth of other proteins that lend themselves to protein complementation, the methods of the present invention can be extended to create any number of distinct SEER systems that are compatibly, each of which is tailored to a distinct sequence. As such, it is possible to simultaneously to detect the presence or absence of multiple sequences within a single sample thus increasing the assay sensitivity, accuracy, and reliability. For example, it is possible to use the aforementioned four GFP variants to simultaneously detect 4 or more specific DNA sequences in a single sample. To this end, any combination and number of specific sequences can be probed simultaneously. Specific mention is made of 2, 3, 4, 5, 6, 7, 8, 9, and 10 distinct sequences, each probed by a specifically designed SEER pair.

In order for the split-protein enzyme that makes up the SEER system to reassemble upon binding to the cognate nucleotide sequence defined by the sequence-specific DNA binding domain contained in the protein for each half to the split-protein enzyme, it is necessary that the cognate sequences to be proximally located. The proximity of these cognate sequences is to be determined on the basis of the placement and number of sequence-specific DNA binding domain, as well as the orientation of the split-protein enzyme within each construct. For example, wherein the first protein contains at least one sequence-specific DNA binding domain and the N-terminal region of the split-protein enzyme and the second protein contains at least one sequence-specific DNA binding domain and the C-terminal region of the split-protein enzyme, then reassembly will only occur if the nucleotide sequence on the DNA to be probed is such that the orientation upon binding of the first and second protein is such that the oligomerization domains are placed in proximity to each other. In the present invention, the functional split-protein reporting enzyme may be reassembled in either orientation (i.e., where the site for the first protein is 5′ or 3′ to the site for the second protein) depending upon the sequence to be identified. Further, in order to accommodate potential steric effects, it is envisioned that the target site for the first protein may be separated from the target site for the second protein by a spacer. Although the spacer length is simply a matter of design choice, preferably lengths range from zero to twenty-five nucleotides, preferably zero, ten, fifteen, and twenty nucleotides. Of course, the present invention also embraces and describes all integers and sub-ranges between zero and twenty-five nucleotides.

Upon reassembly the split-protein enzyme becomes a functional reporter either emitting a fluorescence signal (e.g., GFP) or being capable of performing catalysis (e.g., beta-lactamase).

GFP is a known fluorescent protein having the following biophysicochemical properties—maximal Absorption at 395 nm with a smaller absorbance peak at 470 nm, fluorescence emission spectrum peaks at 509 nm with a shoulder at 540 nm.

In the case of beta-lactamase, the functional enzyme hydrolyzes the substrate nitrocefin. Therefore, upon reassembly, the foregoing properties may be monitored colorimetrically for positive correlation to the presence of the desired nucleotide sequence within a sample comprising a polynucleotide. In addition to the hydrolysis of nitrocefin, many other substrates are available for monitoring of reconstituted beta-lactamase. For example, CCF2 and CCF4 are commercially available fluorescent substrates. CC2 is another fluorescent substrate warranting mention. In addition to being powerful in vitro substrates, CC2, CCF2, and CCF4 may also be used in cell assays. C-mel is a substrate that becomes toxic to eukaryotic cells upon hydrolysis and, as such, this substrate could be used to make SEER perform sequence-dependant cell killing (killing only cells that contain a mutant oncogene or a particular virus, for example). The use of C-mel or other cytotoxic beta-lactamase substrates permits the SEER system to be used in therapeutic methods, which are also embraced by the present application. Another application for the SEER system is in selection assays for modified binding proteins, modified split reporters, etc. by taking advantage of substrates that are toxic to prokaryotic cells that become inactivated by hydrolysis. These beta-lactamase substrates that are toxic to prokaryotic cells that become inactivated by hydrolysis include penicillin, ampicillin, and carbonicillin.

To demonstrate the enablement of the present invention, the following summary is provided of the exemplary data for the present invention. Protein constructs were designed such that the C-terminus of the GFP fragment (1-157) was fused to the N-terminus of Zif268 by means of a 15-residue linker and the N-terminus of the GFP fragment (158-236) was fused to the C-terminus of PBSII through a 15-residue linker. Both protein constructs were incorporated together or separately in the PetDuet vector and verified by DNA sequencing (supplementary material). Protein expression profiles revealed that NGFP-ZnFingerA was expressed in the insoluble fractions, whereas the smaller CGFP-ZnFingerB was expressed in both soluble and insoluble fractions as had been previously observed for coiled-coil peptides appended to similar GFP fragments (14e).

Importantly, no detectable fluorescence was observed in cells expressing each protein alone or together, over a period of 1 week, indicating lack of any detectable non-specific reassembly in the presence of native E. coli DNA. Both proteins, NGFP-ZnFingerA and CGFP-ZnFingerB were separately purified under denaturing conditions utilizing affinity chromatography and characterized by SDS-gel electrophoresis and mass spectrometry (infra).

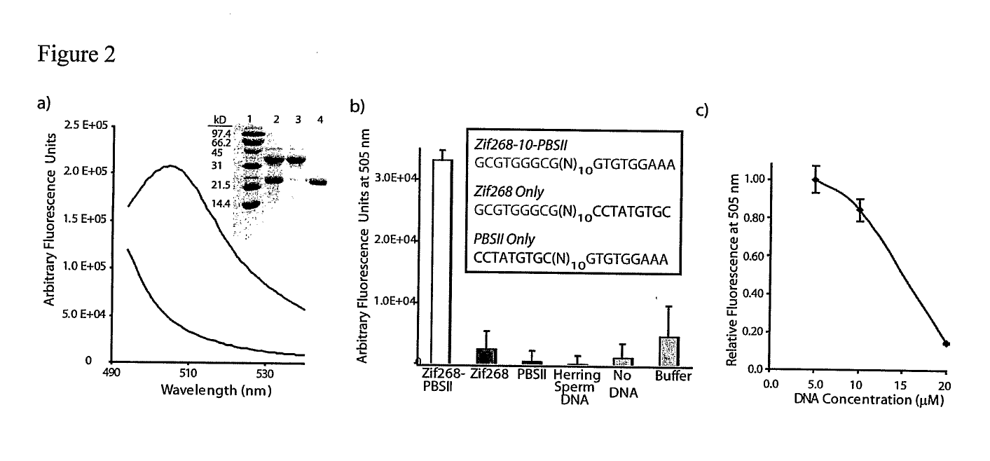

As proof of concept for SEER, the present inventors designed a double-stranded oligonucleotide that contained the two 9-base pair recognition sites for Zif268 and PBSII separated by a 10-nucleotide spacer, Zif268-10-PBSII (15). The 10-nucleotide spacer was designed to allow for both halves of GFP to be juxtaposed on the same face of the target DNA but avoid steric crowding. Equimolar mixtures (15 μM) of the two purified proteins were refolded into 10 mM Tris.HCl, 100 mM NaCl, 1 mM DTT, and 100 μM ZnCl₂ at pH7.5 (buffer A) in the presence or absence of the target oligonucleotide (4 μM). Under these unoptimized conditions the concentration of DNA was 4-fold lower than that of the protein halves, such that the zinc finger tagged GFP halves would not localize to different DNA strands. Fluorescence spectra were acquired 48 hours post-refolding by excitation at 468 nm. Fluorescence emission due to GFP chromophore formation was only observed for samples containing both halves of GFP-zinc-finger fusions in the presence of target oligonucleotide (FIG. 2a), thus strongly supporting our SEER approach.

To further test the sequence specificity of reassembly and subsequent chromophore catalysis, several control experiments were designed. DNA sequences to determine specificity of reassembly consisted of the two half-sites, Zif268 alone, PBSII alone, and non-specific herring sperm DNA. Equimolar mixtures of the two proteins, NGFP-ZnFingerA and CGFP-ZnFingerB, were allowed to refold in the presence of the control and target DNA sequences. No fluorescence was observed in the presence of any of the controls (FIG. 2b), strongly confirming that the reassembly of the two halves of GFP requires the presence of both the zinc finger target sites on a single double stranded DNA template. A final control experiment entailed varying the concentration of the target DNA, with the hypothesis that high molar ratios of the target DNA:dissected proteins would not allow for GFP reassembly as the two halves of GFP would statistically localize to different oligonucleotides with increasing DNA concentrations. The results of this experiment (FIG. 2c) clearly demonstrated that only 4-fold excess of the Zif268-10-PBSII target DNA (20 μM) strongly inhibits GFP (5 μM) reassembly. A first attempt at gauging the effect of spacing of the two DNA target sites also revealed that our designed 10 bp separation between binding sites was substantially better than a 3 bp separation (infra).

Although the foregoing clearly demonstrates the successful DNA templated reassembly of the two fragments of GFP appended to the zinc fingers, Zif268 and PBSII. Additionally, other split-protein enzymes including beta-lactamase (see Examples 5 and 6, 14c) and luciferase (14f), further amplify signal by substrate turnover.

The present invention describes unique constructs that bind nucleic acid. SEER provides an approach for in vivo and in vitro detection of specific DNA sequences, as well as for conditional responses to specific genetic mutations by reassembling proteins that act as cellular toxins. Detection of the signal from reconstituted reporter gene may be done by standard methods known in the art for diagnostic and other detection methods such as fluorescence or calorimetric detection systems. The detection system and sensitivity will vary on the basis of the enzyme to be used in the protein complementation aspect of the SEER system. To this end, the detection system and the required preparatory and monitoring steps would be readily apparent to the skilled artisan.

The SEER system can be easily utilized in a broad range of settings, which is not possible with currently available methods. For example, it is envisioned that the technology of the present application can be used in a settings where bulky equipment or sensitive instrumentation may not be practical. For example, SEER is useful for field detection of specific nucleic acid sequences that are unique to a pathogen, such as for detecting food-borne pathogens or bio terror agents.

Thus, in an embodiment of the present invention, the SEER system can be presented in a kit or prepackaged form that would allow for quick genotype detection in the field where PCR and FISH systems are unavailable. The kit of the present invention contains the components of the SEER system (i.e., the enzymes described herein above). In the kit of the present invention the protein may be in a form selected from frozen, dried (i.e., lyophilized), or aqueous. Additionally, the kit of the present invention preferably contains the reagents for extraction of the biological sample to be tested, a resuspension solution (if necessary), the reaction/hybridization buffer for conducting the complementation assay, and/or a substrate for assaying the presence of a binding event (e.g., nitrocefin, CCF2, CCF4, CC2, C-mel, penicillin, ampicillin, carbonicillin, etc.). In the kit of the present invention, the reaction/hybridization buffer may further contain Zn²⁺ to stabilize the zinc finger domains, when present, in the proteins contained in the kit during the binding assay.

In an embodiment of the present invention, the following pairs of SEER protein pairs are provided: SEQ ID NOs: 14 and 16, SEQ ID NOs: 44 and 46, and SEQ ID NOs: 44 and 48.

In another embodiment of the present invention, the foregoing SEER proteins are individually provided, as well as the polynucleotides encoding the same. In other words, the present invention provides the sequences set forth in SEQ ID NOs: 14, 16, 44, 46, and 48. With respect to the sequences encoding the same, it is well-appreciated from the universal genetic code as to the full range of sequence variants. However, in a preferred embodiment the sequence encoding SEQ ID NOs: 14, 16, 44, 46, and 48 are, SEQ ID NOs: 13, 15, 43, 45, and 47, respectively. The present invention also embraces codon optimized equivalents to the foregoing.

In yet another embodiment of the present invention are proteins having, at least 70%, at least 80%, at least 90%, at least 95%, at least 97.5%, or at least 99% homologous and/or identical to the polypeptides defined above, wherein these proteins have the ability to reconstitute an active fully functional protein when paired with a protein encoding the complementary half of the split-protein enzyme and has the ability to specifically bind to the desired/defined nucleic acid sequence.

In the context of the present application, the polynucleotide sequences defined above may be “homologous” with the defined sequence if at least 70%, preferably at least 80%, more preferably at least 90%, most preferably at least 95% of its base composition and base sequence corresponds to the sequence according to the invention. Further, the homologous polynucleotide should encode a protein meeting the limitations set forth in the paragraph above.

Homology, sequence similarity or sequence identity of nucleotide or amino acid sequences may be determined conventionally by using known software or computer programs such as the BestFit or Gap pairwise comparison programs (GCG Wisconsin Package, Genetics Computer Group, 575 Science Drive, Madison, Wis. 53711). BestFit uses the local homology algorithm of Smith and Waterman, Advances in Applied Mathematics 2: 482-489 (1981) (24), to find the best segment of identity or similarity between two sequences. Gap performs global alignments: all of one sequence with all of another similar sequence using the method of Needleman and Wunsch, J. Mol. Biol. 48:443-453 (1970) (23). When using a sequence alignment program such as BestFit, to determine the degree of sequence homology, similarity or identity, the default setting may be used, or an appropriate scoring matrix may be selected to optimize identity, similarity or homology scores. Similarly, when using a program such as BestFit to determine sequence identity, similarity or homology between two different amino acid sequences, the default settings may be used, or an appropriate scoring matrix, such as blosum45 or blosum80, may be selected to optimize identity, similarity or homology scores.

Within the present invention, the foregoing polynucleotide sequence may be isolated, functionally contained in an expression vector to facilitate expression for in vivo detection and therapeutic methods, or integrated into the host genome to facilitate expression for in vivo detection and therapeutic methods.

The proteins of the present invention that make up the SEER system may be isolated, or expressed in a host cell (e.g., prokaryotic or eukaryotic). With respect to the expressed form, it is envisioned that the protein may be recovered from said host cell by conventional methodologies. Further for in vivo detection and therapeutic methods, the proteins that make up the SEER system may be directly expressed and functionally engaged in the host cell without further purification or processing. In addition, the isolated form of the proteins that make up the SEER system may be delivered into a cell for in vivo detection or therapy. Delivery methods would be readily apparent to the skilled artisan, but liposome-delivery is mentioned by way of example.

The term “isolated” means separated from its natural environment. It is to be understood that the “isolated” polynucleotides and polypeptides of the present invention may further be substantially pure or pure (i.e., the polynucleotides and polypeptides have been purified). As used herein, the term “substantially pure” means that the polynucleotides and polypeptides have been isolated from its natural environment to an extent such that only minor impurities remain (e.g., the resultant polynucleotides and polypeptides are at least 70%, preferably at least 80%, more preferably at least 90%, most preferably at least 95% pure). As used herein, the term “pure” means that the polynucleotides and polypeptides are free from contaminants (i.e., are 100% pure).

The term “polynucleotide” or “nucleic acid sequence” refers in general to polyribonucleotides and polydeoxyribonucleotides, and can denote an unmodified RNA or DNA or a modified RNA or DNA.

The term “polypeptides” is to be understood to mean peptides or proteins, which contain two or more amino acids which are bound via peptide bonds.

The above written description of the invention provides a manner and process of making and using it such that any person skilled in this art is enabled to make and use the same, this enablement being provided in particular for the subject matter of the appended claims, which make up a part of the original description.

As used above, the phrases “selected from the group consisting of,” “chosen from,” and the like include mixtures of the specified materials.

Where a numerical limit or range is stated herein, the endpoints are included. Also, all values and subranges within a numerical limit or range are specifically included as if explicitly written out.

The above description is presented to enable a person skilled in the art to make and use the invention, and is provided in the context of a particular application and its requirements. Various modifications to the preferred embodiments will be readily apparent to those skilled in the art, and the generic principles defined herein may be applied to other embodiments and applications without departing from the spirit and scope of the invention. Thus, this invention is not intended to be limited to the embodiments shown, but is to be accorded the widest scope consistent with the principles and features disclosed herein.

Having generally described this invention, a further understanding can be obtained by reference to certain specific examples, which are provided herein for purposes of illustration only, and are not intended to be limiting unless otherwise specified.

### EXAMPLES

### Example 1

### Cloning of NGFP-Zif268 and CGFP-PBSII Proteins

### General Materials and Methods.

All restriction enzymes, Taq Polymerase, DNA ligase, dNTPS were obtained from New England Biolabs.

### Initial Cloning:

NGFP and CGFP coding DNA sequences were obtained by PCR amplification from plasmids which have been previously described¹ using the following primers to subclone GFP fragments into the pQE30 expression plasmid.

| | |
|---|---|
| NGFP-BamHI: | |
| GCTACGGGATCCATGGCTAGCAAAGGAGAA | (SEQ ID NO: 1) |
| NGFP-PstI: | |
| GCACGTCTGCAGACCTTGTTTGTCTGCCAT | (SEQ ID NO: 2) |
| CGFP-KpnI: | |
| CGTGCAGGTACCAAGAATGGAATCAAAGTG | (SEQ ID NO: 3) |
| CGFP-HindIII: | |
| CGACGTAAGCTTGGATCCTCAGTTGTACAG | (SEQ ID NO: 4) |

NGFP and CGFP fragments were digested with BamHI/PstI and KpnI/HindIII respectively and ligated into pQE30 (Qiagen) expression plasmids containing the Zif268 and PBSII coding regions separated by a flexible 15 amino acid linker, sequences were confirmed by dideoxyoligonucleotide sequencing at the University of Arizona DNA Sequencing Facility.

Protein Expression from pQE30 Plasmids:

Test protein expressions from these two plasmid constructs were unsuccessful in XL1-Blue (Stratagene), Top10 (Invitrogen), and BL21-Gold (DE3) (Novagen) cell lines; consequently a more robust system for protein expression was chosen which would also allow for the expression of both proteins within a single cell. The T7 promoter system (Novagen) contains plasmids specifically designed for this purpose and the present inventors chose the pETDuet-1 expression vector (Novagen) based on previous work which had shown that the expression of the same dissected GFP halves fused to leucine zippers produced adequate yields using a similar pET expression system (13).

Cloning SEER Proteins into pETDuet-1:

Primers were used to amplify the NGFP-Zif268 and CGFP-PBSII genes from the above pQE30 plasmids by PCR amplification and subsequent cloning of the SEER proteins into the pETDuet-1 expression plasmid as well as sequencing primers for both MCS of pETDuet-1 are as follows:

NGFP-Zif268 BglII:

(SEQ ID NO: 5)

CCGCGGCGCGCGCGGAGATCTGATGGCTAGCAAAGGA
NGFP-Zif268 XhoI:

(SEQ ID NO: 6)

CGCGCGCGCGCCGGCTCGAGGTCCTTCTGCCGCAA
CGFP-PBSII EcoRI:

(SEQ ID NO: 7)

CCGCGCGGCCGGCGCGAATTCGGAGAAGCCCTAT
CGFP-PBSII NotI:

(SEQ ID NO: 8)

GGCGGCGCGTGCGGCCGCTTATCAGTTGTACAGTTC
pETDuet-1 MCSI:

(SEQ ID NO: 9)

Fwd-ATGCGTCCGGCGTAGA

(SEQ ID NO: 10)

Rev-GATTATGCGGCCGTGTACAA
pETDuet-1 MCSII:

(SEQ ID NO: 11)

Fwd-TTGTACACGGCCGCATAATC

(SEQ ID NO: 12)

Rev-GCTAGTTATTGCTCAGCGG

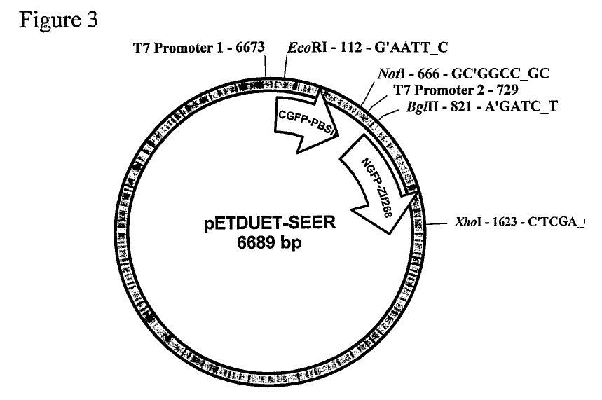

These genes were then successively ligated into the pETDuet-1 expression plasmid using BglII/XhoI and EcoRI/NotI respectively yielding a plasmid containing CGFP-PBSII in MCSI which contains an N-terminal His-tag and NGFP-Zif268 in MCSII which contains a C-terminal Stag this plasmid was called pETDuet-SEER (see FIG. 3). Sequences for CGFP-PBSII and NGFP-Zif268 were confirmed by dideoxyoligonucleotide sequencing at the University of Arizona DNA Sequencing Facility.

The CGFP-PBSII polynucleotide sequence was determined to be that shown in SEQ ID NO: 13, which encodes the amino acid sequence of SEQ ID NO: 14. In the amino acid sequence of SEQ ID NO: 14, amino acid residues 17-100 correspond to PBSII, amino acid residues 101-115 correspond to the linker, and amino acid residues 116-196 of SEQ ID NO: 14 correspond to residues 158 to 238 of GFP.

The NGFP-Zif268 polynucleotide sequence was determined to be that shown in SEQ ID NO: 15, which encodes the amino acid sequence of SEQ ID NO: 16. In the amino acid sequence of SEQ ID NO: 16, amino acid residues 5-165 of SEQ ID NO: 16 correspond to residues 1-157 of GFP, amino acid residues 166-180 correspond to the linker, and amino acid residues 181-267 correspond to Zif268.

### Example 2

### Expression and Purification of NGFP-Zif268 and CGFP-PBSII

### General Materials and Methods.

Buffer A is 10 mM Tris-HCl @ pH=7.5, 100 mM NaCl, 1 mM DTT, and 100 μM ZnCl₂. All reagents were obtained from Sigma unless otherwise noted. LB and 2xYT media were purchased from Becton Dickinson.

### Expression:

BL21-Gold (DE3) cells (Novagen) were transformed with pETDuet-SEER using the standard heat shock protocol, plated on LB-Amp Agar plates, and grown overnight at 37° C. to obtain single colonies. Single colonies were picked and used to inoculate 2xYT media containing Amp and grown overnight with shacking at 37° C. This overnight culture was used to inoculate a one liter 2xYT-Amp culture containing 100 μM ZnCl₂ (EM Science) to a final O.D.₆₀₀ of 0.05. Cells were shaken at 37° C. until an O.D.₆₀₀ of 0.5-0.8 was reached at which time they were induced with 1 mM IPTG (Research Products International Corporation). Cells were induced for three hours after which they were pelleted at 3000 rcf and frozen overnight. This yielded approximately 15 mg of CGFP-PBSII of which 7.5 mg was purified by subsequent IMAC and 26 mg of NGFP-Zif268 of which 6 mg was purified by subsequent IMAC from a one liter culture.

### Purification by IMAC:

Cells were re-suspended in Buffer A and lysed using standard sonication protocols. NGFP-Zif268 was found entirely in inclusion bodies whereas a relatively small amount of CGFP-PBSII was found in the soluble fraction (with the remainder residing in inclusion bodies). Consequently both proteins were purified under denaturing conditions as follows. Inclusion bodies obtained from above were solubilized in Buffer A containing 6 M Urea and incubated on ice for one hour. The resulting solution was diluted to 4 M Urea with Buffer A and clarified by centrifugation at 18,000 rcf for 20 minutes. This lysate was passed over Ni-NTA agarose beads (Qiagen) and eluted with Buffer A containing 4 M Urea and increasing concentrations of imidazole (2, 10, 20, 50, and 500 mM sequentially). NGFP-Zif268 eluted in the 2 mM imidazole fractions whereas CGFP-PBSII eluted in the 50-500 mM fractions (a mixture of both proteins was found in the 10-20 mM fractions). Fractions of CGFP-PBSII which still contained small amounts of NGFP-Zif268 were further purified by dialyzing into Buffer A with 4 M Urea and 2 mM imidazole and re-exposure to the same IMAC column. NGFP-ZIF268 eluted in the flow-through, 2 mM imidazole, whereas CGFP-PBSII eluted in the previously observed fractions.

### Example 3

### Mass Spectroscopy (MALDI) of the SEER Proteins

The refolded SEER proteins were analyzed by MALDI-MS analysis. MALDI mass spectra were acquired on a Bruker Reflex-III MALDI/TOF the masses obtained were within 0.1% of the calculated masses and are shown in Table 1 below.

TABLE 1

| | MH⁺ calculated | MH⁺ found |
|---|---|---|
| NGFP-Zif268 | 32686 | 32648 |
| CGFP-PBSII | 21294 | 21273 |

### Example 4

### Refolding Experiments

### General Materials and Methods.

All spectra were taken on a Photon Technology International (PTI) spectrofluorometer with excitation and emission wavelengths of 468 nm and 505 nm respectively. Slit widths were set to 5 nm for excitation and 10 nm for emission. All refolding experiments were conducted using 3.5K MWCO Slide-A-Lyzer Dialysis Cassettes (Pierce). All DNA constructs used in refolding were obtained HPLC purified from IDT and appear below:

Zif268-3-PBSII:

(SEQ ID NO: 17)

GCGTAGCGTGGGCGTAAGTGTGGAAACACCG
Zif268-10-PBSII:

(SEQ ID NO: 18)

GCGTAGCGTGGGCGTAGGACGATAGTGTGGAAACACCG
Zif268:

(SEQ ID NO: 19)

GCGTAGCGTGGGCGTAGGACGATACCTATGTGCCACCG
PBSII:

(SEQ ID NO: 20)

GCGTACCTATGTGCTAGGACGATAGTGTGGAAACACCG

In the foregoing DNA constructs that were used in the refolding experiments, nucleotides 6-14 of SEQ ID NOs: 17-19 correspond to the Zif268 DNA binding site, nucleotides 25-33 of SEQ ID NO: 19 and nucleotides 6-14 of SEQ ID NO: 20 correspond to the decoy DNA binding site, and nucleotides 18-26 of SEQ ID NO: 17 and nucleotides 25-33 of SEQ ID NOs: 18-20 correspond to the PBSII DNA binding site. The numbers between the zinc finger names indicate the distance between binding sites in base pairs.

Oligos were annealed in 1× BamHI Buffer (NEB) using the following procedure: heating to 95° C. for 7 min, cooling to 56° C. at a rate of 1° C./min, equilibrating at 56° C. for 5 min, and finally cooling to 25° C. at a rate of 1° C./min using a Techne Genius thermocycler. All refolding experiments were conducted at 4° C. The theoretical extinction coefficients for NGFP-Zif268 and CGFP-PBSII at 280 nm are 17210 and 7680 M⁻¹ cm⁻¹ respectively.

### Initial Refolding Experiments:

Initial refolding experiments were conducted using SEER proteins in Buffer A containing 4 M Urea obtained by IMAC from the 10 mM imidazole wash. The concentration of each half in this wash was determined to 15 μM by UV absorbance at 280 nm. Samples were refolded as follows, 4 μM of Zif268-10-PBSII DNA was added to 1 mL of SEER proteins (15 μM each) and dialyzed into Buffer A in a stepwise manner (Buffer A containing 2 M Urea, 1 M Urea, 0.5 M Urea, and twice into Buffer A with no Urea) over a period of two days. A negative control was also performed in which no DNA was added to the SEER proteins. Precipitate was observed in the negative control sample but not in the sample containing Zif268-10-PBSII DNA, indicating that the proteins were only soluble in the presence of DNA (this was confirmed by later observations). Fluorescence excitation and emission spectra of these samples were taken two days after refolding.

### GFP Reassembly as a Function of DNA Concentration:

The concentration of each SEER protein was kept constant at 5 μM while the concentration of Zif268-10-PBSII DNA was varied between 5, 10, and 20 μM in 250 μL total of Buffer A containing 4 M Urea. Samples were refolded as before into Buffer A over a period of two days and emission spectra of each sample were taken two days after refolding.

### Specificity of the GFP Reassembly Process:

Samples containing 5 μM of each SEER protein and 2.5 μM of Zif268-10-PBSII, 2.5 μM of ZIf268, 2.5 μM of PBSII, and 15.4 μg of Herring Sperm (Invitrogen) DNA were prepared in 250 μL total Buffer A containing 4 M Urea. These samples along with a negative control (no DNA) were refolded as described above over a period of two days. Fluorescence emission spectra of each sample were taken two days after refolding.

### Effect of Spacing Between Zinc Finger Binding Sites:

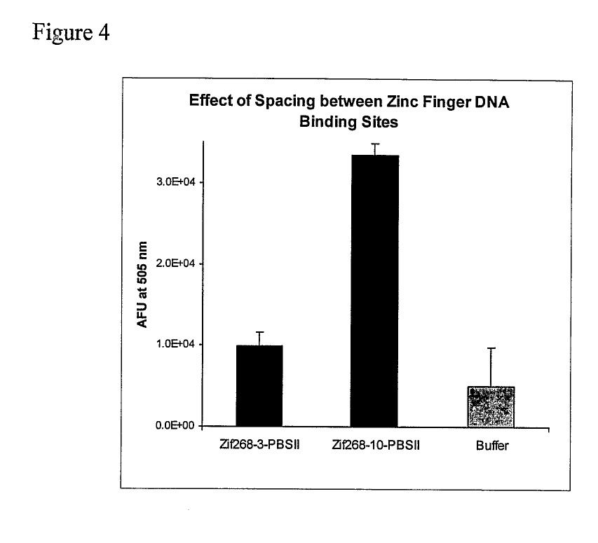

Separate samples containing 5 μM of each SEER protein and 2.5 μM or Zif268-3-PBSII and Zif268-10-PBSII in 250 μL of Buffer A containing 4 M Urea were refolded as stated above over a period of two days. The fluorescence emission spectra of each sample containing DNA with different spacing between binding sites were taken and the results are shown in FIG. 4.

### Example 5

### Cloning, Expressions and Purification of β-Lactamase Based Proteins

### Cloning, Expression, and Purification of the Proteins:

E. coli TEM-1b-lactamase DNA was obtained by PCR using the bacterial expression vector pMAL-c2X (New England Biolabs) as the template. LacA (aa26-aa196) and LacB (aa198-aa290) were cloned into separate pMAL-c2X vectors using standard cloning procedures (vide infra). LacA contained an M182T mutation to enhance the stability of the protein (14c). ZF proteins were constructed by PCR using overlapping primers. Zif268 was cloned C-terminal to LacA, whereas PBSII and PE1A were cloned N-terminal to LacB. The ZF and Lac domains were separated by a 15-aa linker, (GGGGS)₃ (SEQ ID NO: 25). Protein was expressed in BL-21 Star cells (Invitrogen). 100 μM of ZnCl₂ was added to 100 ml of LB growth media. At OD₆₀₀=0.6-0.8, protein expression was induced with 1 mM isopropyl β-D-thiogalactoside (IPTG) for 5 hours at 37° C. Cells were pelleted and resuspended in ZBA (100 nM Tris base, 90 mM KCl, 1 mM MgCl₂, 100 μM ZnCl₂, pH 7.5)/5 mM DTT. The MBP-tagged proteins were purified over amylose columns and eluted in ZBA/5 mM DTT/10 mM maltose, following the methodology of the Protein Fusion and Purification System (New England Biolabs).

LacA portion of β-lactamase was constructed by PCR using 5′-GAGGAGGAGG GATCCCACCCAGAAACGCTGGTG-3′ (SEQ ID NO: 21) as the forward primer and 5′-CTCCTCCTGCAGGCCAGTTAATAGTTTGCGCAACGTTGTTGCCATTGCTACAGGA GTCG-3′ SEQ ID NO: 21) as the reverse primer, using pQE-30 (Qiagen) as the template. The reverse primer carried a mutation that gave an M182T conversion to further stabilize the fold of the peptide. The PCR product was purified over QIAquick PCR purification column (Qiagen). The purified product and a pMAL-c2x plasmid carrying ZnFn Zif268 with N-terminal 15aa linker were digested with PstI and BamHI for 2 hours at 37° C. using NEB Buffer 2 (New England Biolabs). The digested products were visualized on a 1% TAE agarose gel at 100 V for 45 min. The appropriate bands were cut from the gel and DNA was extracted using Montage columns (Millipore). The digested and purified vector and insert were ligated overnight at room temperature with T4 ligase (Promega) in 10 uL reaction volume and 2 uL of the ligation product was transformed into Top 10 cells (Invitrogen).

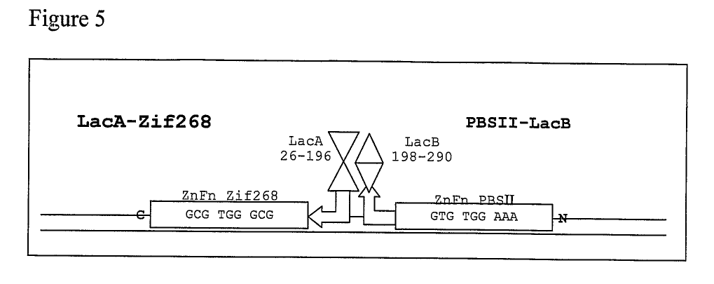

LacB portion of β-lactamase was generated by PCR using 5′-GAGGAGGAGACC GGTGGGGGTGGCGGTTCAGGCGGTGGGGGTTCTGGTGGGGGTGGTACCCTACTT ACTCTAGCTTCCCGGC-3′ (SEQ ID NO: 23) as the forward primer and 5′-CTCCTCCTCAAGCTTCCAATGCTTAATCAGTGAGGC-3′ (SEQ ID NO: 24) as the reverse primer. The forward primer carried a sequence coding for thel 5aa (GGGGS)₃ (SEQ ID NO: 25) linker N-terminal of the LacB. The remaining procedures were similar with the construction of LacA-Zif268 except that LacB was cloned into C-terminal of pMAL-c2x vectors carrying either PBSII or PE1A ZnFn using Agel and HindIII sites. The configuration and orientation of the SEER system is shown in FIG. 5.

The Zif268-LacA polynucleotide sequence was determined to be that shown in SEQ ID NO: 43, which encodes the amino acid sequence of SEQ ID NO: 44. In the amino acid sequence of SEQ ID NO: 44, amino acid residues 3-173 correspond to residues 26-196 of β-lactamase where the Met-182 residue has been replaced with a Thr (with respect to residue numbering in the β-lactamase, please see the discussion below following PE1A-LacB), amino acid residues 174-188 correspond to the linker, and residues 189-286 correspond to ZnFn Zif268. In the Zif268 region of Zif268-LacA, residues 207-213 of SEQ ID NO: 44 correspond to a Zinc finger with a recognition site of GCG, residues 235-241 of SEQ ID NO: 44 correspond to a Zinc finger with a recognition site of TGG, and residues 263-269 of SEQ ID NO: 44 correspond to a Zinc finger with a recognition site of GCG.

The PBS2-LacB polynucleotide sequence was determined to be that shown in SEQ ID NO: 45, which encodes the amino acid sequence of SEQ ID NO: 46. In the amino acid sequence of SEQ ID NO: 46, amino acid residues 5-88 correspond to ZnFn PBS2, amino acid residues 89-103 correspond to the linker, and residues 104-194 correspond to residues 198-290 of β-lactamase (with respect to residue numbering in the β-lactamase, please see the discussion below following PE1A-LacB). In the PBS2 region of PBS2-LacB, residues 19-25 of SEQ ID NO: 46 correspond to a Zinc finger with a recognition site of AAA, residues 47-53 of SEQ ID NO: 46 correspond to a Zinc finger with a recognition site of TGG, and residues 75-81 of SEQ ID NO: 46 correspond to a Zinc finger with a recognition site of GTG.

The PE1A-LacB polynucleotide sequence was determined to be that shown in SEQ ID NO: 47, which encodes the amino acid sequence of SEQ ID NO: 48. In the amino acid sequence of SEQ ID NO: 48, amino acid residues 5-88 correspond to ZnFn PE1A, amino acid residues 89-103 correspond to the linker, and residues 104-194 correspond to residues 198-290 of β-lactamase (with respect to residue numbering in the β-lactamase, please see the discussion below). In the PE1A region of PE1A-LacB, residues 19-25 of SEQ ID NO: 48 correspond to a Zinc finger with a recognition site of AAC, residues 47-53 of SEQ ID NO: 48 correspond to a Zinc finger with a recognition site of AAT, and residues 75-81 of SEQ ID NO: 48 correspond to a Zinc finger with a recognition site of ATA.

It is noted that the residue numbers of amino acids indicated in the foregoing description does not agree with the number of amino acid residues in the indicated sequence for the β-lactamase domains in respective sequences in the sequence listing. The problem lies with the original publication describing the PCA of β-lactamase (Galarneau et al., 2002 Nat. Biotech. 20:619 (ref. 14c). Those authors claimed they obtained a β-lactamase gene coding for 290 aa from plasmid pQE32 from Qiagen. Qiagen's vector description, however, shows this gene to code for only 286 aa. Codon 287 is a stop codon. It appears that Galarneau et al (14c) somehow added 2 aa to the N-term of Qiagen's β-lactamase, and 2 aa to the C-term. Therefore, the numbering below is given to clarify the confusion with respect to the β-lactamase sequence:
According to Qiagen's data, the numbering should be 24-194 (171 aa) for the N-terminal fragment, and 196-286 (91 aa) for the C-terminal fragment.
According to Galarneau et al's data, the numbering is 26-196 (171 aa) for the N-terminal fragment, and 198-290 (93 aa) for the C-terminal fragment.

Although the C-terminal fragment of the present invention has 91 aa, it is described as 198-290 to be consistent with Galarneau et al (14c).

### Protein Design.

SEER-LAC proteins contained two inactive fragments of β-lactamase fused to zinc finger proteins with the ability to recognize specific DNA sequences. The two fragments were designed to bind near each other at adjacent sites in the presence of a user-defined DNA target site to generate a signal. Two 3-finger ZF proteins binding in this way would have the collective capacity to recognize 18 bp of DNA, a target site sufficiently large to be unique in the human genome (27). However, since biologically relevant target sites could not be chosen until the optimal spacer and orientation parameters were established, initial experiments employed designed target sites that were recognized by existing, well-characterized ZF. Zif268 is a naturally occurring 3-finger ZF that has been extensively studied structurally and biochemically (38, 39). It binds the 9 bp sequence 5′-GCG TGG GCG-3′ (SEQ ID NO: 26). PBSII and PE1A are designed 3-finger ZFs assembled from predefined modified ZF domains (1,15), and recognize the sequences 5′-GTG TGG AAA-3′ (SEQ ID NO: 27) and 5′-ATA AAT AAC-3′ (SEQ ID NO: 28), respectively.

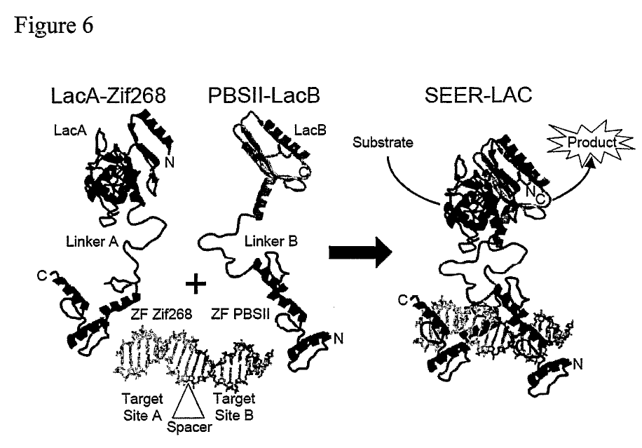

Two inactive fragments of the 290-amino acid TEM1 β-lactamase protein can be generated by splitting the protein between residues 196 and 198 (34). To maintain the correct polarity of the protein fragments, Zif268 was appended to the C-terminus of β-lactamase residues 26-196 (LacA-Zif268; lacking the N-terminal secretory signal sequence), and PBSII or PE1A was appended to the N-terminus of residues 198-290 (PBSII-LacB or PE1A-LacB). The ZF and β-lactamase domains were separated by a 15-aa linker, (GGGGS)₃ (SEQ ID NO: 25, FIG. 6). All proteins were expressed from the vector pMAL-c2X, which additionally appended the 392-amino acid Maltose Binding Protein (MBP) to the N-terminus of all three protein fragments. All experiments were performed with the MBP domain attached.

### Example 6

### Nitrocefin Assay for the Proteins of Example 5

### Nitrocefin Assay.

Hairpin oligonucleotide DNA target Zif268-0-PBSII had the sequence, 5′-GGC TTT CCA CAC CGC CCA CGC GGG TTTT CCC GCG TGG GCG GTG TGG AAA GCC-3′ (SEQ ID NO: 29), and Zif268-0-PE1A had the sequence, 5′-GGC GTT ATT TAT CGC CCA CGC GGG TTTT CCC GCG TGG GCG ATA AAT AAC GCC-3′ (SEQ ID NO: 30), where 5′-CGC TGG GCG-3′ (SEQ ID NO: 31), 5′-GTT TGG AAA-3′ (SEQ ID NO: 32), and 5′-ATA AAT AAC-3′ (SEQ ID NO: 28) are the target sites for ZFs Zif268, PBSII and PE1A, respectively. All oligonucleotides were heated to 95° C. for 10 minutes at 10 μM in ZBA, then slowly cooled to room temperature to form hairpins with a four-thymidine loop. A more complete description of the DNA targets use in this study is offered below.

In a 96-well plate, 120 μL of ZBA was added to the wells followed by 20 μL of 10 μM, 200 nM, 2 nM, or no hairpin oligonucleotide DNA targets (1 μM, 20 nM, 200 pM, 0 final concentration, Operon). 20 μL of 5 μM protein fragments LacA-Zif268 and PBSII-LacB/PE 1A-LacB (0.5 μM final concentration each fragment) was added to the wells before adding 20 μL of 1 mM nitrocefin (0.1 mM final concentration, Calbiochem). All steps were carried out at room temperature. Absorbance at 486 nm was monitored over 20 minutes with a SpectraMax M2 (Molecular Devices). To ensure that trace contamination of the pMAL-C2X-expressed full-length β-lactamase was not contributing to background hydrolysis, samples containing individual SEER fragments at were incubated with substrate. No detectable hydrolysis was observed over the assay interval (data not shown).

### Hairpin Oligonucleotide DNA Target Design.

Hairpin oligonucleotide DNA targets used in this study had the general sequence shown below, where X1aX1aX1a is a three nucleotide subsite for zinc finger 1, and X1a′X1a′X1a′ is its complement. A 4 nt hairpin was formed by four thymidines. Between the 9 bp binding sites for the two zinc finger proteins was spacer of 0, 6 or 10 bp, indicated as (N)_{spacer}. The full sequence of all target site DNAs used in this study are shown in Table 2 below. For simplicity, only the top strand (3′ end of the hairpin oligonucleotide) is shown.

TABLE 2
Full sequence¹ of hairpin oligonucleotide DNA targets

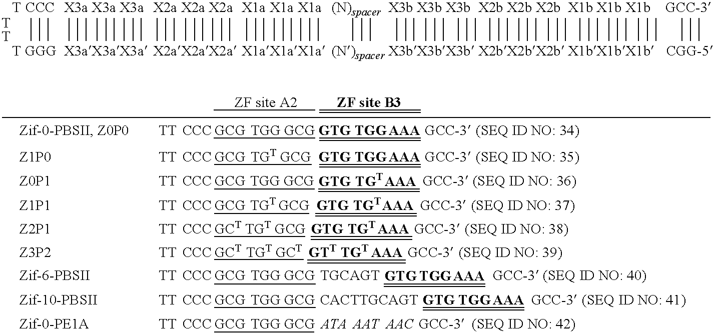

¹For simplicity, only the top strand (3′ end of the hairpin oligonucleotide) is shown.
²Zif268 target site is underlined, G->T mutations shown in superscript
³PBSII target site shown in bold with double underline, PE1A target site shown in italics.

### DNA-Dependent Enzyme Activity.

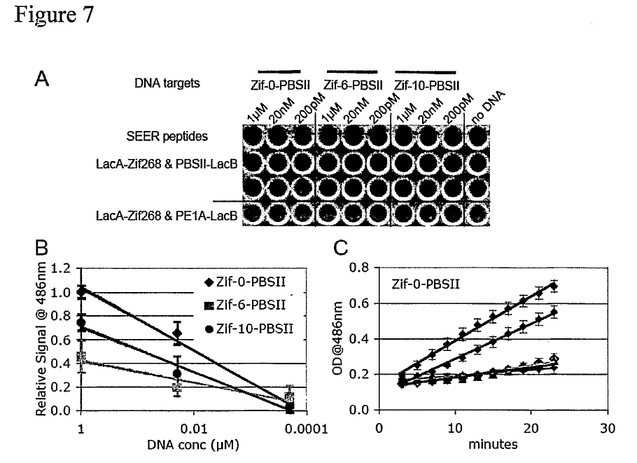

β-lactamase activity assays were conducted using the colorimetric substrate nitrocefin, which changes from yellow to red (486 nm) upon hydrolysis. Based on similar studies with chimeric ZF-endonucleases, the present inventors expected the spacing between the two ZF sites (“spacer”) on the target DNA to be crucial for efficient enzyme reassembly. To examine this relationship, nitrocefin assays were performed in triplicate with 0.5 μM of each LacA-Zif268 and PBSII-LacB proteins in the presence of hairpin oligonucleotides containing the two target sites at spacer lengths of 0, 6, and 10 bp (labeled Zif-0-PBSII, Zif-6-PBSII, and Zif-10-PBSII in FIG. 7), at concentrations of 1 μM, 20 nM, 200 pM, and no DNA as a control. DNA-assisted enzyme reassembly was shown with all three spacer lengths (FIG. 7A). The hydrolysis rates were the highest for the 0 bp spacer, followed by 10 and 6 bp (FIG. 7B). On the target with the 0 bp spacer, hydrolysis rates were proportional to DNA concentration (25 mU/min at 1 μM DNA, 20 mU/min at 20 nM, 7 mU/min at 200 pM) with a R² correlation coefficient of 0.963 (FIG. 7C).

A background hydrolysis rate around 5 mU/min was observed for the negative control of LacA-Zif268 paired with PE1A-LacB on the above target DNAs. A difference in signal intensity between the 1 μM DNA sample and background was clearly distinguishable by the earliest time point in our assay (3 minutes), and became more pronounced over time. All hydrolysis rates were linear over the 23 minute assay interval, with correlation coefficients greater than 0.99 (FIG. 7D).

### Effects of Target Site Mutations on the SEER Signal Intensity.

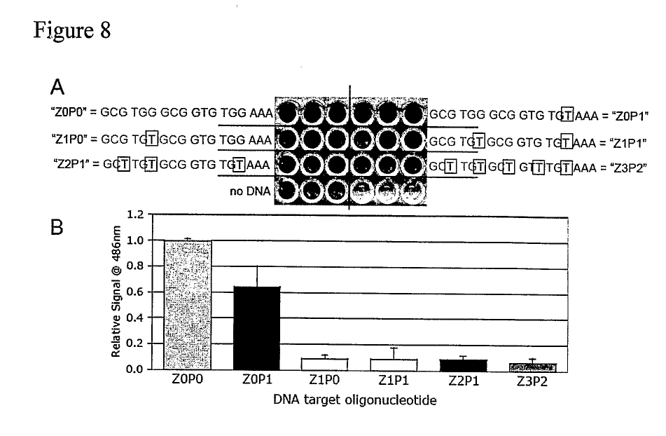

In order to determine the sensitivity of SEER to mutations, nitrocefin assays were performed using oligonucleotide targets carrying different mutations on either one or both of the ZF binding sites (FIG. 8). At 1 μM DNA concentration and 0.5 μM each protein, a single mutation in the Zif268 target site reduced enzyme activity to essentially background levels. A single base pair mutation in the PBSII target site resulted in a 28% reduction in the hydrolysis rate. Target sites carrying two or more mutations lowered the signal to the levels comparable to background.

### SEER Binding Domains are Interchangeable.

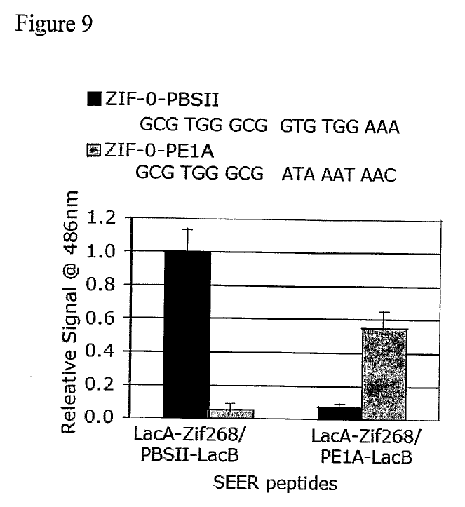

In order to demonstrate the generality the SEER-LAC to target significantly different binding sites, a nitrocefin assay was performed with two different DNA target sequences, one carrying Zif268 and PBSII target sites with no spacer (Zif-0-PBSII) and the other carrying Zif268 and PELA target sites with no spacer (Zif-0-PE1A). Both SEER combinations reassembled in the presence of their cognate DNA sequences (FIG. 9). Inappropriate target DNA produced a signal similar to the background signal of no target DNA.

### SEER Binding in the Presence of Genomic DNA.

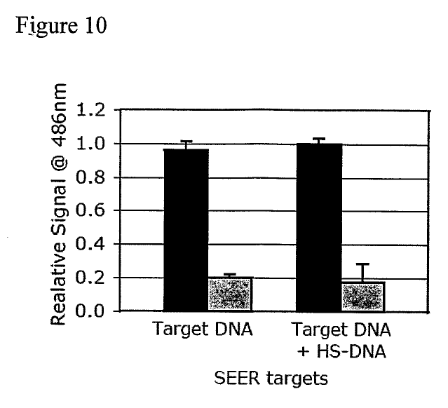

The previous experiments were conduct with purified DNA targets. However, some applications of this technology might require it to recognize its target in the presence of complex DNA, such as a genome, which might contain multiple alternative sites for the individual SEER proteins. To investigate if the presence of complex double-stranded DNA would interfere with this assay, a nitrocefin assay was performed in the presence or absence of herring sperm DNA (HSDNA). The concentration of HS-DNA used was equimolar in base pairs (i.e., equal in mass) to 1 μM of the oligonucleotide target DNA. Under these conditions, there was no difference in relative signal intensity when 0.5 μM each of LacA-Zif268 and PBSII-LacB proteins were incubated with 1 μM of Zif-0-PBSII target DNA in the presence or absence of HS-DNA (FIG. 10, black bars). As a negative control, the proteins were also incubated with the Zif-0-PE1A target DNA (FIG. 10, white bars). Although the relative signal generated using this target was somewhat higher than in previous assays, there was essentially no change in the signal intensity in the presence of HS-DNA.

### Example 7

### Cloning, Expression, and Purification of MBD2 Based Proteins

### General Materials and Methods:

All enzymes were obtained from NEB, dNTP's were purchasd from Fermentas. A pUC57 plasmid containing an optimized E. coli gene encoding for human MBD2 (48,49) (residues 147-215) was designed and subsequently obtained from GeneScript.

### MBD2 Cloning:

The MBD2 insert was obtained via PCR amplification from the pUC57 vector using the following primers. This insert was used to replace a zinc finger, which was fused to CGFP, with MBD2 in a construct which was previously described. (50)

| | |
|---|---|
| MBD2-EcoRI: | |
| GCGTATGAATTCGGAAAGCGGCAAACGC | (SEQ ID NO: 49) |
| MBD2-AgeI: | |
| CGGTTAACCGGTCATTTTGCCGGTACG | (SEQ ID NO: 50) |

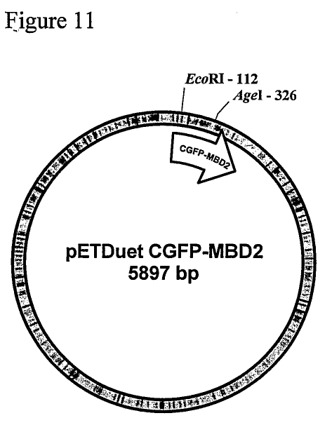

The MBD2 insert was sequentially digested with EcoRI and AgeI. The existing pETDuet CGFP-zinc-finger vector was also sequentially digested and treated with Antartic Phosphatase to prevent re-ligation of the zinc-finger coding region, which would yield the original plasmid. The MBD2 insert was ligated into the doubly digested vector using a 1:10 molar ratio of vector: insert. This yielded a CGFP-MBD2 fusion, which was separated by a flexible 15 amino acid linker, sequences were confirmed by dideoxyoligonucleotide sequencing at the University of Arizona DNA Sequencing Facility. A map of this plasmid is shown below (FIG. 11).

### NGFP-Zif268 Cloning:

An NGFP-Zif268 construct was cloned as described in Example 1.

### Sequence Analysis:

The CGFP-MBD2 polynucleotide sequence was determined to be that shown in SEQ ID NO: 51, which encodes the amino acid sequence of SEQ ID NO: 52. In the amino acid sequence of SEQ ID NO: 52, amino acid residues 17-85 correspond to the MBD2 domain, amino acid residues 88-102 correspond to the linker, and amino acid residues 103-183 of SEQ ID NO: 14 correspond to residues 158 to 238 of GFP.

The NGFP-Zif268 polynucleotide sequence was determined to be that shown in SEQ ID NO: 15, which encodes the amino acid sequence of SEQ ID NO: 16. In the amino acid sequence of SEQ ID NO: 16, amino acid residues 5-165 of SEQ ID NO: 16 correspond to residues 1-157 of GFP, amino acid residues 166-180 correspond to the linker, and amino acid residues 181-267 correspond to Zif268.

### Expression and Purification of CGFP-MBD2 and NGFP-Zif268:

General Materials and Methods: Buffer A is 10 mM Tris-HCl @ pH=7.5, 100 mM NaCl, 1 mM DTT, and 100 μM ZnCl₂. All reagents were obtained from Research Products International Corporation unless otherwise noted. LB-Agar and 2xYT media were purchased from Becton Dickinson.

Expression of CGFP-MBD2: Electrocompetent BL21-Gold (DE3) cells (Novagen) were transformed with the pETDuet CGFP-MBD2 plasmid using standard protocols, plated on LB-Amp Agar plates, and grown overnight at 37° C. to obtain single colonies. Single colonies were picked and used to inoculate 2xYT media containing Amp (100 μg/mL) and grown overnight with shacking at 37° C. This overnight culture was used to inoculate a one-liter 2xYT-Amp culture containing 100 μM ZnCl₂ (EM Science) to a final O.D.₆₀₀ of 0.05. Cells were shaken at 37° C. until an O.D.₆₀₀ of 1.32 was reached at which time they were induced with 1 mM IPTG. Cells were induced for three hours after which they were pelleted at 4000 rcf and frozen overnight. This yielded approximately 10 mg of CGFP-MBD2 of which 5.8 mg was purified by subsequent IMAC.

Purification of CGFP-MBD2 by IMAC: Cells were re-suspended in Buffer A and lysed using standard sonication protocols and clarified for 30 minutes at 18,000 rcf. CGFP-MBD2 was found predominantly in the soluble fraction. This lysate was passed over Ni-NTA agarose beads (Qiagen) and eluted with Buffer A containing increasing concentrations of imidazole (2, 10, 20, 50, and 500 mM sequentially). CGFP-MBD2 eluted in the 50-500 mM imidazole fractions. Fractions containing CGFP-MBD2 were found to have high concentrations of DNA (as determine by the A₂₆₀/A₂₈₀), therefore CGFP-MBD2 was further purified under denaturing conditions. CGFP-MBD2 obtained above was diluted into an equivalent volume of Buffer A containing 8 M Urea (4 M Urea final). This sample was re-exposed to Ni-NTA agarose beads and the protein was eluted with Buffer A containing 4 M Urea and increasing concentrations of imidazole (2, 10, 20, 50, and 500 mM sequentially). Fractions containing CGFP-MBD2 were pooled, concentrated, and dialyzed into Buffer A containing 4 M Urea. Concentrations were obtained using protein absorbance measurements at 280 nm (ε=14440 M⁻¹ cm⁻¹).

Expression and Purification of NGFP-Zif268: NGFP-Zif268 was expressed and purified as described previously³. Concentrations were obtained using protein absorbance measurements at 280 nm (ε=17210 M⁻¹ cm⁻¹).

### Example 8

### Characterization of the SEER Proteins

### SDS-PAGE:

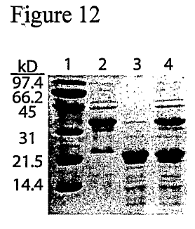

Equivalent amounts of purified NGFP-Zif268 (32.7 kD) and CGFP-MBD2 (19.6 kD) were loaded on a 15% SDS-PAGE gel (FIG. 12).

### MALDI:

Samples of the refolded mCpG-SEER proteins from above were sent for MALDI-MS analysis. MALDI mass spectra were acquired on a Bruker Reflex-III MALDI/TOF the masses obtained are shown below.

NGFP-Zif268 MH⁺ calculated is 32686; found: 32648

CGFP-MBD2 MH⁺ calculated is 19590; found: 19572

### Example 9

### Refolding Experiments

### General Materials and Methods:

All spectra were taken on a Photon Technology International spectrofluorometer with excitation and emission wavelengths of 468 nm and 505 nm respectively. Slit widths were set to 5 nm for excitation and 10 nm for emission. All refolding experiments were conducted using 3.5 kD MWCO Slide-A-Lyzer Dialysis Cassettes (Pierce) unless otherwise noted. All DNA constructs used in refolding are shown in Figure S5 and were obtained HPLC purified from IDT. Oligos were annealed in 1× BamHI Buffer (NEB) using the following procedure: heating to 95° C. for 7 min, cooling to 56° C. at a rate of 1° C./min, equilibrating at 56° C. for 5 min, and finally cooling to 25° C. at a rate of 1° C./min using a Techne Genius thermocycler. All refolding experiments were conducted at 4° C. in uncovered chambers.

### Initial Refolding Experiments:

Samples were refolded as follows, 2.5 μM of mCpG-Zif268 DNA was added to 5 μM NGFP-Zif268 and 20 μM CGFP-MBD2 in Buffer A containing 4 M Urea in a total volume of 250 μL. This sample was dialyzed into Buffer A in a stepwise manner (Buffer A containing 2 M Urea, 1 M Urea, 0.5 M Urea, and twice into Buffer A with no Urea) over a period of two days. A negative control was also performed in which no DNA was added to the mCpG-SEER proteins. Fluorescence excitation and emission spectra of these samples were taken two days after refolding.

mCpG-SEER Specificity:

Individual samples containing 5 μM NGFP-Zif268 and 20 μM CGFP-MBD2 plus 2.5 μM of each separate control DNA sequence (below) along with a sample containing an equivalent amount (11.9 μg) of Herring Sperm DNA (Invitrogen) and a negative control with no DNA were prepared at a final volume of 250 μL in Buffer A containing 4 M Urea. Samples were refolded as before into Buffer A over a period of two days and emission spectra of each sample were taken two days after refolding. Background fluorescence from Buffer A and the negative control (No DNA) at 505 nm were subtracted sequentially from all readings. Fluorescence at 505 nm for each sample was made relative to the mCpG-Zif268 sample. This experiment was repeated and the relative fluorescence values were averaged in.

Specificity substrates:
mCpG-Zif268:

(SEQ ID NO: 51)

5′-GCGTAₘCGTAGGACGATACGCCCACGCCACCG
3′-CGCATGCₘATCCTGCTATGCGGGTGCGGTGGC
CpG-Zif268:

(SEQ ID NO: 52)

5′-GCGTACGTAGGACGATACGCCCACGCCACCG
3′-CGCATGCATCCTGCTATGCGGGTGCGGTGGC
mCpG Only:

(SEQ ID NO: 53)

5′-GCGTAₘCGTAGGACGATAGCACATAGGCACCG
3′-CGCATGCₘATCCTGCTATCGTGTATCCGTGGC
mCpG-Zif268 G to T:

(SEQ ID NO: 54)

5′-GCGTAₘCGTAGGACGATACGCACACGCCACCG
3′-CGCATGCₘATCCTGCTATGCGTGTGCGGTGGC

In the foregoing DNA constructs used in the refolding experiments, the bold text indicates the MBD2 site, the underlined text indicates the Zif268 site, and the italics text indicates mutation sites.
Effect of DNA Target Site Spacing on mCpG-SEER:

Individual samples containing 5 μM NGFP-Zif268 and 20 μM CGFP-MBD2 plus 2.5 μM of each separate control DNA sequence with different spacings between target sites (3, 6, 10, and 13 b.p. below) were prepared at a final volume of 250 μL in Buffer A containing 4 M Urea. These samples along with a negative control (no DNA) were refolded as described above over a period of two days. Emission spectra of each sample were taken two days after refolding. Spectra were background subtracted using the no DNA sample and were plotted relative to the sample containing the 10 bp spacing. This experiment was repeated using 10 kD MWCO Slide-A-Lyzer Dialysis Cassettes (Pierce) and averaged in order to obtain trends based on relative fluorescence values.

Spacing substrates:
3:

(SEQ ID NO: 55)

5′-GCGTAₘCGTAGCGCCCACGCCACCG
3′-CGCATGCₘATCGCGGGTGCGGTGGC
6:

(SEQ ID NO: 56)

5′-GCGTAₘCGTAGGACCGCCCACGCCACCG
3′-CGCATGCₘATCCTGGCGGGTGCGGTGGC
10:

(SEQ ID NO: 57)

5′-GCGTAₘCGTAGGACGATACGCCCACGCCACCG
3′-CGCATGCₘATCCTGCTATGCGGGTGCGGTGGC
13:

(SEQ ID NO: 58)

5′-GCGTAₘCGTAGGACGATAACCCGCCCACGCCACCG
3′-CGCATGCₘATCCTGCTATTGGGCGGGTGCGGTGGC

In the foregoing DNA constructs used in the refolding experiments, the bold text indicates the MBD2 site and the underlined text indicates the Zif268 site.

### Example 10

### Effect of Target Site Spacing on SEER-GFP

### General Materials and Methods:

All spectra were taken on a Photon Technology International spectrofluorometer with excitation and emission wavelengths of 468 nm and 505 nm respectively. Slit widths were set to 5 nm for excitation and 10 nm for emission. All refolding experiments were conducted using 3.5 KD MWCO Slide-A-Lyzer Dialysis Cassettes (Pierce). All DNA constructs used in refolding are shown in Figure S6 and were obtained HPLC purified from IDT. Oligos were annealed in 1× BamHI Buffer (NEB) using the following procedure: heating to 95° C. for 7 min, cooling to 56° C. at a rate of 1° C./min, equilibrating at 56° C. for 5 min, and finally cooling to 25° C. at a rate of 1° C./min using a Techne Genius thermocycler. All refolding experiments were conducted at 4° C. in uncovered chambers. Duplicate experiments were compared by the use of an internal standard, 5(6)-carboxyflourescein (FAM), obtained from Sigma prepared at 20 nM in Buffer A. FAM emission spectra were acquired by excitation at 490 nm. SEER-GFP data from duplicate experiments were normalized relative to FAM emission at 512 nm.

```
DNA constructs used in the refolding experiments
to test the effect of spacing of SEER-GFP:
  0 (SEQ ID NO: 59):
GCGTAGCGTGGGCGGTGTGGAAACACCG
  3 (SEQ ID NO: 60):
GCGTAGCGTGGGCGTAAGTGTGGAAACACCG
  6 (SEQ ID NO: 61):
GCGTAGCGTGGGCGTTAGTCGTGTGGAAACACCG
10 (SEQ ID NO: 62):
GCGTAGCGTGGGCGTAGGACGATAGTGTGGAAACACCG
13 (SEQ ID NO: 63):
GCGTAGCGTGGGCGTTAGTCACTAGAGGTGTGGAAACACCG
16 (SEQ ID NO: 64):
GCGTAGCGTGGGCGTTAGTCACTAGAGGACGTGTGGAAACACCG
20 (SEQ ID NO: 65):
GCGTAGCGTGGGCGTTAGTCACTAGAGGACGATAGTGTGGAAAACACCG
```

In the foregoing constructs, the bold text indicates the Zif268 site and the underlined text indicate PBSII sites. Numbers indicate the distance between binding sites in base pairs.

### Sensitivity of SEER-GFP to Target Site Spacing:

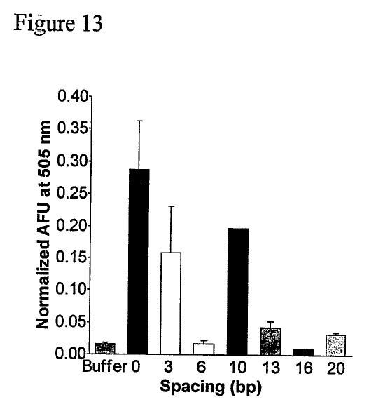

Spectra were acquired from samples which contained 5 μM NGFP-Zif268, 5 μM CGFP-PBSII, and 2.5 μM of each target DNA. Spectra were taken four days post-refolding and were normalized to the final DNA concentration after dialysis (using the absorbance at 260 nm) and then to the 20 nM FAM emission (internal standard). Refolding experiments were repeated, separately, and the data are plotted below (FIG. 13).

Numerous modifications and variations on the present invention are possible in light of the above teachings. It is, therefore, to be understood that within the scope of the accompanying claims, the invention may be practiced otherwise than as specifically described herein.

### REFERENCES

1.—Segal, D. J. (2002). The use of zinc finger peptides to study the role of specific factor binding sites in the chromatin environment. Methods 26, 76-83.
2. Beerli, R. R., Segal, D. J., Dreier, B., and Barbas III, C. F. (1998). Toward controlling gene expression. at will: specific regulation of the erbB-2/HER-2 promoter by using polydactyl zinc finger proteins constructed from modular building blocks. Proc Natl Acad Sci USA 95, 14628-14633.
3. Segal, D. J., Dreier, B., Beerli, R. R., and Barbas III, C. F. (1999). Toward controlling gene expression at will: selection and design of zinc finger domains recognizing each of the 5′-GNN-3′ DNA target sequences. Proc Natl Acad Sci USA 96, 2758-2763.
4. Dreier, B., Beerli, R. R., Segal, D. J., Flippin, J. D., and Barbas IIi, C. F: (2001). Development of zinc finger domains for recognition of the 5′-ANN-3′ family of D14A sequences and their use in the construction of artificial transcription factors. J Biol Chem 276, 29466-29478.
5. Remy I, Michnick S W. A cDNA library functional screening strategy based on. fluorescent protein complementation assays to identify novel components of signaling pathways. Methods. 2004 April; 32(4):381-8.
6. Michnick S W, Remy I, Cknpbell-Valois F X, Vallee-Belisle A, Pelletier J N. Detection of protein-protein interactions by protein fragment complementation strategies. Methods Enzymol. 2000; 328:208-30.
7. Pelletier J N, Arndt K M, Pluckthun A, Michnick S W. An in vivo library-versus-library selection of optimized protein-protein interactions. Nat. Biotechnol. 1999 July; 17(7):683-90.
8. Pabo, C. O. et al. (1982) Nature 298, 443-447.
9. Bibikova M, Carroll D, Segal D J, Trautman J K, Smith J, Kim Y G, Chandrasegaran S. Stimulation of homologous recombination through targeted cleavage by chimeric nucleases. Mol Cell Biol. 2001 January; 21(1):289-97.
10. Bibikova M, Beumer K, Trautman J K, Carroll D. Enhancing gene targeting with designed zinc finger nucleases. Science. 2003 May 2; 300(5620):764.
11. Lloyd A, Plaisier C L, Carroll D, Drews G N. Targeted mutagenesis using zinc-finger nucleases in Arabidopsis. Proc Natl Acad Sci USA. 2005 Feb. 8; 102(6):2232-7.
12. Urnov F D, Miller J C, Lee Y L, Beausejour C M, Rock J M, Augustus S, Jamieson A C, Porteus M H, Gregory P D, Holmes M C. Highly efficient endogenous human. gene correction using designed zinc-finger nucleases. Nature. 2005 Apr. 3.
13. Patikoglou, G.; Burley, S. K. Annu. Rev. Biophys. Biomolec. Struct. 1997, 26, 289-325.
14. a) Johnsson, N.; Varshavsky, A. Proc. Natl. Acad. Sci. U.S.A. 1994, 91, 10340-10344. b) Rossi, F.; Charlton, C. A.; Blau, H. M. Proc. Natl. Acad. Sci. U.S.A. 1997, 94, 8405-8410. c) Galarneau, A.; Primeau, M.; Trudeau, L. E.; Michnick, S. W. Nat. Biotechnol. 2002, 20, 619-622. d) Pelletier, J. N.; Campbell-Valois, F. X.; Michnick, S. W. Proc. Natl. Acad. Sci. U.S.A. 1998, 95, 12141-12146. e) Ghosh, I.; Hamilton, A. D.; Regan, L. J. Am. Chem. Soc. 2000, 122, 5658-5659. f) Hu, C. D.; Kerppola, T. K. Nat. Biotechnol. 2003, 21, 539-545. g) Paulmurugan, R.; Gambhir, S. S. Anal. Chem. 2003, 75, 1584-1589. h) Sugimoto, K.; Mori, Y.; Makino, K.; Ohkubo, K.; Morii, T. J. Am. Chem. Soc. 2003, 125, 5000-5004.
15. Blancafort, P.; Segal, D. J.; Barbas, C. F., 3rd Mol. Pharmacol. 2004, 66, 1361-1371.
16. Pavletich, N. P.; Pabo, C. O, Science 1991, 252, 809-817.
17. Magliery, T. J.; Wilson, C. G. M.; Pan, W. L.; Mishler, D.; Ghosh, I.; Hamilton, A. D.; Regan, L. J. Am. Chem. Soc. 2005, 127, 146-157.
18. a) Paul, N.; Joyce, G. F. Curr. Opin. Chem. Biol. 2004, 8, 634-639. b) Ghosh, I.; Chmielewski, J. Curr. Opin. Chem. Biol. 2004, 8, 640-644. c) Calderone, C. T.; Liu, D. R. Curr. Opin. Chem. Biol. 2004, 8, 645-653.
19. Venter, J. C., Adams, M. D., Myers, E. W., Li, P. W., Mural, R. J., Sutton, G. G., Smith, H. O., Yandell, M., Evans, C. A., Holt, R. A., et al. (2001) Science 291, 1304-51.
20. Yang L. & et al. (2005) Biochemistry, 44, 7469-7478.
21. Montclare J. K. et al. (2003) J. Am. Chem. Soc., 125, 3416.
22. Padmanabhan, S. et al. (1997) Biochemistry 36, 6424-6436.
23. Needleman and Wunsch, J. Mol. Biol. 48:443-453 (1970).
24. Smith and Waterman, Advances in Applied Mathematics 2: 482-489 (1981).
25. Ray, P., Pimenta, H., Paulmurugan, R., Berger, F., Phelps, M. E., Iyer, M., and Gambhir, S. S. (2002) Proc Natl Acad Sci USA 99, 3105-10.
26. Paulmurugan, R., Umezawa, Y., and Gambhir, S. S. (2002) Proc Natl Acad Sci USA 99, 15608-13.
27. Segal, D. J., Beerli, R. R., Blancafort, P., Dreier, B., Effertz, K., Huber, A., Koksch, B., Lund, C. V., Magnenat, L., Valente, D., and Barbas III, C. F. (2003) Biochemistry 42, 2137-2148.
28. Segal, D., and Barbas III, C. F. (2001) Curr. Opin. Biotech. 12, 632-637.
29. Liu, Q., Xia, Z., Zhong, X., and Case, C. C. (2002) J Biol Chem 277, 3850-6.
30. Lee, D. K., Seol, W., and Kim, J. S. (2003) Curr Top Med Chem 3, 645-57.
31. Klug, A. (2005) FEBS Lett 579, 892-4.
32. Pabo, C. O.; Peisach, E.; Grant, R. A. Annu. Rev. Biochem. 2001, 70, 313-340.
33. Huang, G. S. et al. (1989) J. Mol. Biol. 205, 189-200.
34. Jamieson, A. C., Miller, J. C., and Pabo, C. O. (2003) Nat Rev Drug Discov 2, 361-8.
35. Carroll, D. (2004) Methods Mol Biol 262, 195-207.
36. Tan, W., Zhu, K., Segal, D. J., Barbas, C. F., 3rd, and Chow, S. A. (2004) J Virol 78, 1301-13.
37. Stains, C. I., Porter, J. R., Ooi, A. T., Segal, D. J., and Ghosh, I. (2005) J Am Chem Soc 127, 10782-3.
38. Elrod-Erickson, M., Rould, M. A., Nekludova, L., and Pabo, C. O. (1996) Structure 4, 1171-1180.
39. Wolfe, S. A., Nekludova, L., and Pabo, C. O. (2000) Annu Rev Biophys Biomol Struct 29, 183-212.
40. Neri, D. et al. (1992) J. Mol. Biol. 223, 743-767.
41. Mondragon, A. et al. (1989) J. Mol. Biol. 205, 179-188.
42. Sevilla-Sierra, P. et al. (1994) J. Mol. Biol. 235, 1003-1020.
43. Zlokarnik, G., Negulescu, P. A., Knapp, T. E., Mere, L., Burres, N., Feng, L., Whitney, M., Roemer, K., and Tsien, R. Y. (1998) Science 279, 84-8.
44. Queenan, A. M., Foleno, B., Gownley, C., Wira, E., and Bush, K. (2004) J Clin Microbiol 42, 269-75.
45. Tan, S., Guschin, D., Davalos, A., Lee, Y. L., Snowden, A. W., Jouvenot, Y., Zhang, H. S., Howes, K., McNamara, A. R., Lai, A., Ullman, C., Reynolds, L., Moore, M., Isalan, M., Berg, L. P., Campos, B., Qi, H., Spratt, S. K., Case, C. C., Pabo, C. O., Campisi, J., and Gregory, P. D. (2003) Proc Natl Acad Sci USA 100, 11997-2002.
46. Choo, Y., and Klug, A. (1994) Proc. Natl. Acad. Sci. U.S.A. 91, 11168-72.
47. (a) Hendrich, B.; Bird, A. Mol. Cell. Biol. 1998, 18, 6538-6547. (b) Hendrich, B.; Abbott, C.; McQueen, H.; Chambers, D.; Cross, S.; Bird, A. Mam. Gen. 1999, 10, 906-912.
48. Hendrich, B. et al. Genomic structure and chromosomal mapping of the murine and human Mbd1, Mbd2, Mbd3, and Mbd4 genes. Mammalian Genome 10, 906-912 (1999).
49. Hendrich, B. & Bird, A. Identification and characterization of a family of mammalian methyl-CpG binding proteins. Molecular and Cellular Biology 18, 6538-6547 (1998).
50. Stains, C. I., Porter, J. R., Ooi, A. T., Segal, D. J. & Ghosh, I. DNA sequence-enabled reassembly of the green fluorescent protein. Journal of the American Chemical Society 127, 10782-10783 (2005).

1. A nucleotide sequence detection system comprising:
a first protein wherein said protein comprises at least sequence-specific DNA binding domain and the N-terminal oligomerization domain of a split-protein enzyme, wherein said at least one zinc finger domain is separated from said N-terminal oligomerization domain of said split-protein enzyme by a linker; and
a second protein wherein said protein comprises at least sequence-specific DNA binding domain and the C-terminal oligomerization domain of said split-protein enzyme, wherein said at least one zinc finger domain is separated from said C-terminal oligomerization domain of said split-protein enzyme by a linker.

2. The nucleotide sequence detection system of claim 1, wherein said sequence-specific DNA binding domain is selected from the group consisting of a helix-turn-helix protein, a miniature DNA binding protein, a methyl-cytosine binding domain, and a zinc finger domain.

3. The nucleotide sequence detection system of claim 2, wherein at least one of said first protein and said second protein contains at least one methyl-cytosine binding domain as said sequence-specific DNA binding domain.

4. The nucleotide sequence detection system of claim 1, wherein at least one of said first protein and said second protein contains at least one zinc finger domain as said sequence-specific DNA binding domain.

5. The nucleotide sequence detection system of claim 1, wherein each of said first protein and said second protein contain at least one zinc finger domain as said sequence-specific DNA binding domain.

6. The nucleotide sequence detection system of claim 5, wherein said at least one zinc finger domain of said first protein is contained within a zinc finger module which is derived from a zinc finger protein selected from the group consisting of Zif268, PBSII and PE1A.

7. The nucleotide sequence detection system of claim 5, wherein said at least one zinc finger domain of said second protein is contained within a zinc finger module which is derived from a zinc finger protein selected from the group consisting of Zif268, PBSII and PE1A.

8. The nucleotide sequence detection system of claim 5, wherein said at least one zinc finger domain of said first protein are located C-terminal to the N-terminal oligomerization domain of said split-protein enzyme.

9. The nucleotide sequence detection system of claim 5, wherein said at least one zinc finger domain of said second protein are located N-terminal to the C-terminal oligomerization domain of said split-protein enzyme.

10. The nucleotide sequence detection system of claim 1, wherein said split-protein enzyme reassembles to form a functional enzyme;
wherein said first protein binds the cognate nucleotide sequence for the sequence-specific DNA biding domain comprised therein,
wherein said second protein binds the cognate nucleotide sequence for the sequence-specific DNA biding domain comprised therein, and
wherein the cognate nucleotide sequence for said first protein is located 5′ to the cognate nucleotide sequence for said second protein.

11. The nucleotide sequence detection system of claim 10, wherein said split-protein enzyme is selected from the group consisting of beta-galactosidase, beta-lactamase, dihydrofolate reductase, green fluorescent protein, and luciferase, and variants or homologs thereof.

12. The nucleotide sequence detection system of claim 10, wherein said split-protein enzyme is a beta-lactamase, variants or homologs thereof.

13. The nucleotide sequence detection system of claim 10, wherein said split-protein enzyme is green fluorescent protein, variants or homologs thereof.

14. The nucleotide sequence detection system of claim 1, wherein said linker in said first protein ranges from 0 to 30 amino acids.

15. The nucleotide sequence detection system of claim 1, wherein said linker in said first protein is 15 amino acids.

16. The nucleotide sequence detection system of claim 1, wherein said linker in said second protein ranges from 0 to 30 amino acids.

17. The nucleotide sequence detection system of claim 1, wherein said linker in said second protein is 15 amino acids.

18. The nucleotide sequence detection system of claim 1, wherein said first protein has the sequence comprising SEQ ID NO: 16 and said second protein has the sequence comprising SEQ ID NO: 14.

19. The nucleotide sequence detection system of claim 1, wherein said first protein has the sequence comprising SEQ ID NO: 46 and said second protein has the sequence comprising SEQ ID NO: 44.

20. The nucleotide sequence detection system of claim 1, wherein said first protein has the sequence comprising SEQ ID NO: 48 and said second protein has the sequence comprising SEQ ID NO: 44.

21. The nucleotide sequence detection system of claim 1, wherein said first protein has the sequence comprising SEQ ID NO: 16 and said second protein has the sequence comprising SEQ ID NO: 52.

22. An isolated polynucleotide encoding said first protein of the nucleotide sequence detection system of claim 1.

23. The isolated polynucleotide of claim 22, wherein said polynucleotide is selected from the group consisting of SEQ ID NO: 15, SEQ ID NO: 45, and SEQ ID NO: 47.

24. An isolated polynucleotide encoding said second protein of the nucleotide sequence detection system of claim 1.

25. The isolated polynucleotide of claim 24, wherein said polynucleotide is selected from the group consisting of SEQ ID NO: 13, SEQ ID NO: 43, and SEQ ID NO: 51.

26. A kit comprising the nucleotide sequence detection system of claim 1 and a hybridization buffer.

27. The kit of claim 26, wherein said first protein and said second protein are in a lyophilized form.

28. A method of detecting the presence of a specific nucleotide sequence in a sample comprising a polynucleotide, wherein said method comprises:
contacting said sample with the nucleotide sequence detection system of claim 1 for a time and under conditions suitable to facilitate hybridization, wherein said nucleotide sequence detection system is tuned to detect said specific nucleotide sequence by the arrangement and number of sequence-specific DNA binding domains contained within said first protein and said second protein;
monitoring the formation of activity associated with the split-protein enzyme when in a reassembled state; and
correlating an observed positive activity from said monitoring to the presence of said specific sequence in said polynucleotide.

29. The method of claim 28, wherein said split-protein enzyme is green fluorescent protein and said monitoring comprises monitoring the fluorescence emission at 509 nm upon excitation at 395 nm.

30. The method of claim 28, wherein said split-protein enzyme is beta-lactamase and said monitoring comprises monitoring hydrolysis of a substrate selected from the group consisting of nitrocefin, CCF2, CCF4, CC2, C-mel, penicillin, ampicillin, and carbonicillin.

31. The method of claim 28, wherein said method is a method of detecting a genetic abnormality in a subject in need thereof.

32. The method of claim 28, wherein said method is a method of detecting single nucleotide polymorphism in a subject in need thereof.

33. The method of claim 28, wherein said method is a method of detecting shortening of telomeres in a subject in need thereof.

34. The method of claim 33, wherein said subject in need thereof is a subject having or suspected of having cancer.

35. The method of claim 33, wherein said subject in need thereof is a subject having or suspected of having an age related disease.

36. The method of claim 28, wherein said method is a method of determining the age of cells or cloned animals and said specific nucleotide sequence is the repeat sequence in telomeres.

37. The method of claim 28, wherein said method is a method of diagnosing cancer in a subject in need thereof and said specific nucleotide sequence is a unique marker for a specific type of cancer.

38. The method of claim 28, wherein said method is a method of identifying an infectious agent and said sample is selected from the group consisting of a tissue sample, a blood sample, a sera sample, a nasal swab, a vaginal swab, and a rectal swab.

39. The method of claim 28, wherein said method is a method of identifying an infectious agent and said sample is selected from the group consisting of food, beverage, and water.

40. The method of claim 28, wherein said method is a sample-to-source matching method wherein the specific nucleotide sequence represents a unique nucleotide sequence obtained from a biological sample of interest and said sample is obtained from a subject suspected to contain said unique nucleotide sequence.

41. The method of claim 40, wherein said biological sample of interest is selected from the group consisting of blood, hair, skin, sperm, and semen.

42. The method of claim 40, wherein said sample is selected from the group consisting of blood, hair, skin, sperm, and semen.

43. A method of treating eradicating a viral infection in a subject in need thereof, comprising:
tailoring the sequence specificity of said sequence-specific DNA binding domains of said nucleotide sequence detection system of claim 1 to the virus infecting said subject to a unique nucleic acid sequence thereto, wherein said split-protein enzyme facilitates hydrolysis of a substrate that becomes toxic to said virus upon hydrolysis;
administering an effective amount of said nucleotide sequence detection system of claim 1 to said subject; and
administering an effective amount of said substrate to said subject.

44. The method of claim 43, wherein said split-protein enzyme is beta-lactamase.

45. The method of claim 44, wherein said substrate is C-mel.

46. A method of treating cancer in a subject in need thereof, comprising:
tailoring the sequence specificity of said sequence-specific DNA binding domains of said nucleotide sequence detection system of claim 1 to a mutant oncogene in said subject to a unique nucleic acid sequence thereto, wherein said split-protein enzyme facilitates hydrolysis of a substrate that becomes toxic to said virus upon hydrolysis;
administering an effective amount of said nucleotide sequence detection system of claim 1 to said subject; and
administering an effective amount of said substrate to said subject.

47. The method of claim 46, wherein said split-protein enzyme is beta-lactamase.

48. The method of claim 47, wherein said substrate is C-mel.

49. A method of detecting the presence of specific sites of DNA methylation within a specific sequence of a polynucleotide of a subject in need thereof comprising:
tailoring the sequence specificity of said sequence-specific DNA binding domains of said nucleotide sequence detection system of claim 1 to a specific DNA sequence in said subject to a unique nucleic acid sequence thereto, wherein said sequence-specific DNA binding domain of at least one of said first protein and said second protein is a methyl binding domain;
delivering an effective amount of said nucleotide sequence detection system of claim 1 to a sample obtained from said subject;
monitoring the formation of activity associated with the split-protein enzyme when in a reassembled state; and
correlating an observed positive activity from said monitoring to the presence of DNA methylation within said specific sequence in said polynucleotide.

50. The method of claim 49, wherein said methyl binding domain is a methyl-cytosine binding domain.

51. The method of claim 50, wherein said first protein has the sequence comprising SEQ ID NO: 16 and said second protein has the sequence comprising SEQ ID NO: 52.

52. The method of claim 49, wherein the presence of said DNA methylation is correlated with a propensity for or a diagnosis of cancer.

53. A method for simultaneous detection the presence of multiple specific nucleotide sequences in a sample comprising a polynucleotide, wherein said method comprises:
contacting said sample with two or more different nucleotide sequence detection systems of claim 1 for a time and under conditions suitable to facilitate hybridization, wherein said nucleotide sequence detection systems are tuned to detect independent specific nucleotide sequences by the arrangement and number of sequence-specific DNA binding domains contained within said first protein and said second protein and wherein said split-protein enzyme for each nucleotide sequence detection system is distinct from any other,
monitoring the formation of activity associated with the split-protein enzymes when in a reassembled state; and
correlating an observed positive activity from said monitoring to the presence of said specific sequences in said polynucleotide.

54. The method of claim 53, wherein said wherein at least one of said split-protein enzymes is selected from the group consisting of beta-galactosidase, beta-lactamase, dihydrofolate reductase, green fluorescent protein, and luciferase, and variants or homologs thereof.

55. The method of claim 53, wherein at least one of said split-protein enzymes is a beta-lactamase, variants or homologs thereof.

56. The method of claim 53, wherein at least one of said split-protein enzymes is green fluorescent protein, variants or homologs thereof.

57. The method of claim 56, wherein at least one of said split-protein enzymes is selected from the group consisting of green fluorescent protein, cyan fluorescent protein, yellow fluorescent protein, red fluorescent protein, and reef coral fluorescent protein.

58. The method of claim 53, wherein said contacting is with three to five of said nucleotide sequence detection systems.


SEQUENCE LISTING (76 sequences)

SEQ ID NO: 1 - DNA, 30 bp, Artificial Sequence
  Description of Artificial Sequence Synthetic oligonucleotide
gctacgggatccatggctagcaaaggagaa

SEQ ID NO: 2 - DNA, 30 bp, Artificial Sequence
  Description of Artificial Sequence Synthetic oligonucleotide
gcacgtctgcagaccttgtttgtctgccat

SEQ ID NO: 3 - DNA, 30 bp, Artificial Sequence
  Description of Artificial Sequence Synthetic oligonucleotide
cgtgcaggtaccaagaatggaatcaaagtg

SEQ ID NO: 4 - DNA, 30 bp, Artificial Sequence
  Description of Artificial Sequence Synthetic oligonucleotide
cgacgtaagcttggatcctcagttgtacag

SEQ ID NO: 5 - DNA, 37 bp, Artificial Sequence
  Description of Artificial Sequence Synthetic oligonucleotide
ccgcggcgcgcgcggagatctgatggctagcaaagga

SEQ ID NO: 6 - DNA, 35 bp, Artificial Sequence
  Description of Artificial Sequence Synthetic oligonucleotide
cgcgcgcgcgccggctcgaggtccttctgccgcaa

SEQ ID NO: 7 - DNA, 34 bp, Artificial Sequence
  Description of Artificial Sequence Synthetic oligonucleotide
ccgcgcggccggcgcgaattcggagaagccctat

SEQ ID NO: 8 - DNA, 36 bp, Artificial Sequence
  Description of Artificial Sequence Synthetic oligonucleotide
ggcggcgcgtgcggccgcttatcagttgtacagttc

SEQ ID NO: 9 - DNA, 16 bp, Artificial Sequence
  Description of Artificial Sequence Synthetic oligonucleotide
atgcgtccggcgtaga

SEQ ID NO: 10 - DNA, 20 bp, Artificial Sequence
  Description of Artificial Sequence Synthetic oligonucleotide
gattatgcggccgtgtacaa

SEQ ID NO: 11 - DNA, 20 bp, Artificial Sequence
  Description of Artificial Sequence Synthetic oligonucleotide
ttgtacacggccgcataatc

SEQ ID NO: 12 - DNA, 19 bp, Artificial Sequence
  Description of Artificial Sequence Synthetic oligonucleotide
gctagttattgctcagcgg

SEQ ID NO: 13 - DNA, 591 bp, Artificial Sequence
  Description of Artificial Sequence Synthetic polynucleotide
atgggcagcagccatcaccatcatcaccacagccaggatccgaattcggagaagccctatgcttgtccggaatgtggtaagtccttcagccagcgcgcaaacctgcgcgcccaccagcgtacccacacgggtgaaaaaccgtataaatgcccagagtgcggcaaatcttttagccgcagcgatcacctgactacccatcaacgcactcatactggcgagaagccatacaaatgtccagaatgtggcaagtctttcagtcgcagcgatgtgctggtgcgccaccaacgtactcacaccggtgggggtggcggttcaggcggtgggggttctggtgggggtggtaccaagaatggaatcaaagtgaacttcaagacccgccacaacattgaagatggaagcgttcaactagcagaccattatcaacaaaatactccaattggcgatggccctgtccttttaccagacaaccattacctgtccacacaatctgccctttcgaaagatcccaacgaaaagagagaccacatggtccttcttgagtttgtaacagctgctgggattacacatggcatggatgaactgtacaactga

SEQ ID NO: 14 - protein, 196 aa, Artificial Sequence
  Description of Artificial Sequence Synthetic construct
MGSSHHHHHHSQDPNSEKPYACPECGKSFSQRANLRAHQRTHTGEKPYKCPECGKSFSRSDHLTTHQRTHTGEKPYKCPECGKSFSRSDVLVRHQRTHTGGGGGSGGGGSGGGGTKNGIKVNFKTRHNIEDGSVQLADHYQQNTPIGDGPVLLPDNHYLSTQSALSKDPNEKRDHMVLLEFVTAAGITHGMDELYN

SEQ ID NO: 15 - DNA, 882 bp, Artificial Sequence
  Description of Artificial Sequence Synthetic polynucleotide
atggcagatctgatggctagcaaaggagaagaactcttcactggagttgtcccaattcttgttgaattagatggtgatgttaacggccacaagttctctgtcagtggagagggtgaaggtgatgcaacatacggaaaacttaccctgaagttcatctgcactactggcaaactgcctgttccatggccaacactagtcactactctgtgctatggtgttcaatgcttttcaagatacccggatcatatgaaacggcatgactttttcaagagtgctatgcccgaaggttatgtacaggaaaggaccatcttcttcaaagatgacggcaactacaagacacgtgctgaagtcaagtttgaaggtgatacccttgttaatagaatcgagttaaaaggtattgacttcaaggaagatggcaacattctgggacacaaattggaatacaactataactcacacaacgttcccatcatggcagacaaacaaggtctgcagggcggttcaggcggtgggggttctggcgggggtgggtaccccggggaacgcccttacgcttgcccagtggagtcctgtgatcgccgcttctcccgctccgacgagctcacccgccacatccgcatccacacaggccagaagcccttccagtgccgcatctgcatgcgcaacttcagccgcagcgaccacctcaccacccacatccgcacccacacaggcgaaaagccctttgcctgcgacatctgtggaagaaagtttgccaggagcgatgaacgcaagaggcataccaagatccacttgcggcagaaggacctcgagtctggtaaagaaaccgctgctgcgaaatttgaacgccagcacatggactcgtctactagcgcagcttaa

SEQ ID NO: 16 - protein, 293 aa, Artificial Sequence
  Description of Artificial Sequence Synthetic construct
MADLMASKGEELFTGVVPILVELDGDVNGHKFSVSGEGEGDATYGKLTLKFICTTGKLPVPWPTLVTTLCYGVQCFSRYPDHMKRHDFFKSAMPEGYVQERTIFFKDDGNYKTRAEVKFEGDTLVNRIELKGIDFKEDGNILGHKLEYNYNSHNVPIMADKQGLQGGSGGGGSGGGGYPGERPYACPVESCDRRFSRSDELTRHIRIHTGQKPFQCRICMRNFSRSDHLTTHIRTHTGEKPFACDICGRKFARSDERKRHTKIHLRQKDLESGKETAAAKFERQHMDSSTSAA

SEQ ID NO: 17 - DNA, 31 bp, Artificial Sequence
  Description of Artificial Sequence Synthetic oligonucleotide
gcgtagcgtgggcgtaagtgtggaaacaccg

SEQ ID NO: 18 - DNA, 38 bp, Artificial Sequence
  Description of Artificial Sequence Synthetic oligonucleotide
gcgtagcgtgggcgtaggacgatagtgtggaaacaccg

SEQ ID NO: 19 - DNA, 38 bp, Artificial Sequence
  Description of Artificial Sequence Synthetic oligonucleotide
gcgtagcgtgggcgtaggacgatacctatgtgccaccg

SEQ ID NO: 20 - DNA, 38 bp, Artificial Sequence
  Description of Artificial Sequence Synthetic oligonucleotide
gcgtacctatgtgctaggacgatagtgtggaaacaccg

SEQ ID NO: 21 - DNA, 33 bp, Artificial Sequence
  Description of Artificial Sequence Synthetic primer
gaggaggagggatcccacccagaaacgctggtg

SEQ ID NO: 22 - DNA, 59 bp, Artificial Sequence
  Description of Artificial Sequence Synthetic primer
ctcctcctgcaggccagttaatagtttgcgcaacgttgttgccattgctacaggagtcg

SEQ ID NO: 23 - DNA, 82 bp, Artificial Sequence
  Description of Artificial Sequence Synthetic primer
gaggaggagaccggtgggggtggcggttcaggcggtgggggttctggtgggggtggtaccctacttactctagcttcccggc

SEQ ID NO: 24 - DNA, 36 bp, Artificial Sequence
  Description of Artificial Sequence Synthetic primer
ctcctcctcaagcttccaatgcttaatcagtgaggc

SEQ ID NO: 25 - protein, 15 aa, Artificial Sequence
  Description of Artificial Sequence Synthetic peptide
GGGGSGGGGSGGGGS

SEQ ID NO: 26 - DNA, 9 bp, Artificial Sequence
  Description of Artificial Sequence Synthetic oligonucleotide
gcgtgggcg

SEQ ID NO: 27 - DNA, 9 bp, Artificial Sequence
  Description of Artificial Sequence Synthetic oligonucleotide
gtgtggaaa

SEQ ID NO: 28 - DNA, 9 bp, Artificial Sequence
  Description of Artificial Sequence Synthetic oligonucleotide
ataaataac

SEQ ID NO: 29 - DNA, 52 bp, Artificial Sequence
  Description of Artificial Sequence Synthetic oligonucleotide
ggctttccacaccgcccacgcgggttttcccgcgtgggcggtgtggaaagcc

SEQ ID NO: 30 - DNA, 52 bp, Artificial Sequence
  Description of Artificial Sequence Synthetic oligonucleotide
ggcgttatttatcgcccacgcgggttttcccgcgtgggcgataaataacgcc

SEQ ID NO: 31 - DNA, 9 bp, Artificial Sequence
  Description of Artificial Sequence Synthetic oligonucleotide
cgctgggcg

SEQ ID NO: 32 - DNA, 9 bp, Artificial Sequence
  Description of Artificial Sequence Synthetic oligonucleotide
gtttggaaa

SEQ ID NO: 33 - DNA, 18 bp, Artificial Sequence
  Description of Artificial Sequence Synthetic oligonucleotide
gcgtgggcgataaataac

SEQ ID NO: 34 - DNA, 26 bp, Artificial Sequence
  Description of Artificial Sequence Synthetic oligonucleotide
ttcccgcgtgggcggtgtggaaagcc

SEQ ID NO: 35 - DNA, 26 bp, Artificial Sequence
  Description of Artificial Sequence Synthetic oligonucleotide
ttcccgcgtgtgcggtgtggaaagcc

SEQ ID NO: 36 - DNA, 26 bp, Artificial Sequence
  Description of Artificial Sequence Synthetic oligonucleotide
ttcccgcgtgggcggtgtgtaaagcc

SEQ ID NO: 37 - DNA, 26 bp, Artificial Sequence
  Description of Artificial Sequence Synthetic oligonucleotide
ttcccgcgtgtgcggtgtgtaaagcc

SEQ ID NO: 38 - DNA, 26 bp, Artificial Sequence
  Description of Artificial Sequence Synthetic oligonucleotide
ttcccgcttgtgcggtgtgtaaagcc

SEQ ID NO: 39 - DNA, 26 bp, Artificial Sequence
  Description of Artificial Sequence Synthetic oligonucleotide
ttcccgcttgtgctgtttgtaaagcc

SEQ ID NO: 40 - DNA, 32 bp, Artificial Sequence
  Description of Artificial Sequence Synthetic oligonucleotide
ttcccgcgtgggcgtgcagtgtgtggaaagcc

SEQ ID NO: 41 - DNA, 36 bp, Artificial Sequence
  Description of Artificial Sequence Synthetic oligonucleotide
ttcccgcgtgggcgcacttgcagtgtgtggaaagcc

SEQ ID NO: 42 - DNA, 26 bp, Artificial Sequence
  Description of Artificial Sequence Synthetic oligonucleotide
ttcccgcgtgggcgataaataacgcc

SEQ ID NO: 43 - DNA, 867 bp, Artificial Sequence
  Description of Artificial Sequence Synthetic polynucleotide
ggatcccacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgactcctgtagcaatggcaacaacgttgcgcaaactattaactggcctgcagggcggttcaggcggtgggggttctggtgggggtggtacccccggggagaagccctacgcttgcccagtggagtcctgtgatcgccgcttctcccgctccgacgagctcacccgccacatccgcatccacacaggccagaagcccttccagtgccgcatctgcatgcgcaacttcagccgcagcgaccacctcaccacccacatccgcacccacacaggcgaaaagcccttcgcctgcgacatctgtggaagaaagtttgccaggagcgatgaacgcaagaggcataccaagatccacaccggtgagcagaagcttatctctgaagaagaccagtgaaagctt

SEQ ID NO: 44 - protein, 286 aa, Artificial Sequence
  Description of Artificial Sequence Synthetic construct
GSHPETLVKVKDAEDQLGARVGYIELDLNSGKILESFRPEERFPMMSTFKVLLCGAVLSRIDAGQEQLGRRIHYSQNDLVEYSPVTEKHLTDGMTVRELCSAAITMSDNTAANLLLTTIGGPKELTAFLHNMGDHVTRLDRWEPELNEAIPNDERDTTTPVAMATTLRKLLTGLQGGSGGGGSGGGGTPGEKPYACPVESCDRRFSRSDELTRHIRIHTGQKPFQCRICMRNFSRSDHLTTHIRTHTGEKPFACDICGRKFARSDERKRHTKIHTGEQKLISEEDQ

SEQ ID NO: 45 - DNA, 588 bp, Artificial Sequence
  Description of Artificial Sequence Synthetic polynucleotide
ggatcccccggggagaagccctatgcttgtccggaatgtggtaagtccttcagtcagcgcgcaaacctgcgcgcccaccagcgtacccacacgggtgaaaaaccgtataaatgcccagagtgcggcaaatcttttagccgcagcgatcacctgaccacccatcaacgcactcatactggcgagaagccatacaaatgtccagaatgtggcaagtctttctcccgcagcgatgtgctggtgcgccaccaacgtactcacaccggtgggggtggcggttcaggcggtgggggttctggtgggggtggtaccctacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggaagctt

SEQ ID NO: 46 - protein, 196 aa, Artificial Sequence
  Description of Artificial Sequence Synthetic construct
GSPGEKPYACPECGKSFSQRANLRAHQRTHTGEKPYKCPECGKSFSRSDHLTTHQRTHTGEKPYKCPECGKSFSRSDVLVRHQRTHTGGGGGSGGGGSGGGGTLLTLASRQQLIDWMEADKVAGPLLRSALPAGWFIADKSGAGERGSRGIIAALGPDGKPSRIVVIYTTGSQATMDERNRQIAEIGASLIKHWKL

SEQ ID NO: 47 - DNA, 588 bp, Artificial Sequence
  Description of Artificial Sequence Synthetic polynucleotide
ggatcccccggggagaagccctatgcttgtccggaatgtggtaagtccttcagcgatagcggcaacctgcgcgtgcaccagcgtacccatacgggtgaaaaaccgtataaatgcccagagtgcggcaaatctttcagtaccactggcaacctgaccgtgcatcaacgcacccacactggcgagaagccatacaaatgtccagaatgtggcaagtccttctctcagaaaagctccctgatcgcccaccaacgtactcacaccggtgggggtggcggttcaggcggtgggggttctggtgggggtggtaccctacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggaagctt

SEQ ID NO: 48 - protein, 196 aa, Artificial Sequence
  Description of Artificial Sequence Synthetic construct
GSPGEKPYACPECGKSFSDSGNLRVHQRTHTGEKPYKCPECGKSFSTTGNLTVHQRTHTGEKPYKCPECGKSFSQKSSLIAHQRTHTGGGGGSGGGGSGGGGTLLTLASRQQLIDWMEADKVAGPLLRSALPAGWFIADKSGAGERGSRGIIAALGPDGKPSRIVVIYTTGSQATMDERNRQIAEIGASLIKHWKL

SEQ ID NO: 49 - DNA, 28 bp, Artificial Sequence
  Description of Artificial Sequence Synthetic primer
gcgtatgaattcggaaagcggcaaacgc

SEQ ID NO: 50 - DNA, 27 bp, Artificial Sequence
  Description of Artificial Sequence Synthetic primer
cggttaaccggtcattttgccggtacg

SEQ ID NO: 51 - DNA, 31 bp, Artificial Sequence
  Description of Artificial Sequence Synthetic oligonucleotide
gcgtacgtaggacgatacgcccacgccaccg

SEQ ID NO: 52 - DNA, 31 bp, Artificial Sequence; identical to SEQ ID NO: 51
  Description of Artificial Sequence Synthetic oligonucleotide

SEQ ID NO: 53 - DNA, 31 bp, Artificial Sequence
  Description of Artificial Sequence Synthetic oligonucleotide
gcgtacgtaggacgatagcacataggcaccg

SEQ ID NO: 54 - DNA, 31 bp, Artificial Sequence
  Description of Artificial Sequence Synthetic oligonucleotide
gcgtacgtaggacgatacgcacacgccaccg

SEQ ID NO: 55 - DNA, 24 bp, Artificial Sequence
  Description of Artificial Sequence Synthetic oligonucleotide
gcgtacgtagcgcccacgccaccg

SEQ ID NO: 56 - DNA, 27 bp, Artificial Sequence
  Description of Artificial Sequence Synthetic oligonucleotide
gcgtacgtaggaccgcccacgccaccg

SEQ ID NO: 57 - DNA, 31 bp, Artificial Sequence; identical to SEQ ID NO: 51
  Description of Artificial Sequence Synthetic oligonucleotide

SEQ ID NO: 58 - DNA, 34 bp, Artificial Sequence
  Description of Artificial Sequence Synthetic oligonucleotide
gcgtacgtaggacgataacccgcccacgccaccg

SEQ ID NO: 59 - DNA, 28 bp, Artificial Sequence
  Description of Artificial Sequence Synthetic oligonucleotide
gcgtagcgtgggcggtgtggaaacaccg

SEQ ID NO: 60 - DNA, 31 bp, Artificial Sequence; identical to SEQ ID NO: 17
  Description of Artificial Sequence Synthetic oligonucleotide

SEQ ID NO: 61 - DNA, 34 bp, Artificial Sequence
  Description of Artificial Sequence Synthetic oligonucleotide
gcgtagcgtgggcgttagtcgtgtggaaacaccg

SEQ ID NO: 62 - DNA, 38 bp, Artificial Sequence; identical to SEQ ID NO: 18
  Description of Artificial Sequence Synthetic oligonucleotide

SEQ ID NO: 63 - DNA, 41 bp, Artificial Sequence
  Description of Artificial Sequence Synthetic oligonucleotide
gcgtagcgtgggcgttagtcactagaggtgtggaaacaccg

SEQ ID NO: 64 - DNA, 44 bp, Artificial Sequence
  Description of Artificial Sequence Synthetic oligonucleotide
gcgtagcgtgggcgttagtcactagaggacgtgtggaaacaccg

SEQ ID NO: 65 - DNA, 48 bp, Artificial Sequence
  Description of Artificial Sequence Synthetic oligonucleotide
gcgtagcgtgggcgttagtcactagaggacgatagtgtggaaacaccg

SEQ ID NO: 66 - DNA, 28 bp, Artificial Sequence
  Description of Artificial Sequence Synthetic oligonucleotide
gcgtgggcgnnnnnnnnnngtgtggaaa

SEQ ID NO: 67 - DNA, 28 bp, Artificial Sequence
  Description of Artificial Sequence Synthetic oligonucleotide
gcgtgggcgnnnnnnnnnncctatgtgc

SEQ ID NO: 68 - DNA, 28 bp, Artificial Sequence
  Description of Artificial Sequence Synthetic oligonucleotide
cctatgtgcnnnnnnnnnngtgtggaaa

SEQ ID NO: 69 - DNA, 18 bp, Artificial Sequence
  Description of Artificial Sequence Synthetic oligonucleotide
gcgtgggcggtgtggaaa

SEQ ID NO: 70 - DNA, 18 bp, Artificial Sequence
  Description of Artificial Sequence Synthetic oligonucleotide
gcgtgggcggtgtgtaaa

SEQ ID NO: 71 - DNA, 18 bp, Artificial Sequence
  Description of Artificial Sequence Synthetic oligonucleotide
gcgtgtgcggtgtggaaa

SEQ ID NO: 72 - DNA, 18 bp, Artificial Sequence
  Description of Artificial Sequence Synthetic oligonucleotide
gcgtgtgcggtgtgtaaa

SEQ ID NO: 73 - DNA, 18 bp, Artificial Sequence
  Description of Artificial Sequence Synthetic oligonucleotide
gcttgtgcggtgtgtaaa

SEQ ID NO: 74 - DNA, 18 bp, Artificial Sequence
  Description of Artificial Sequence Synthetic oligonucleotide
gcttgtgctgtttgtaaa

SEQ ID NO: 75 - DNA, 18 bp, Artificial Sequence
  Description of Artificial Sequence Synthetic oligonucleotide
gcgtgggcggtgtggaaa

SEQ ID NO: 76 - DNA, 72 bp, Artificial Sequence
  Description of Artificial Sequence Synthetic oligonucleotide
ggcnnnnnnnnnnnnnnnnnnnnnnnnnnnngggttttcccnnnnnnnnnnnnnnnnnnnnnnnnnnnngcc